(19) United States
(12) Patent Application Publication
(10) Pub. No.: US 2023/0279347 A1
(43) Pub. Date: Sep. 7, 2023

(54) USE OF FATE MODULATORS TO IMPROVE NERVE REGENERATION

(71) Applicant: The Board of Regents of the University of Texas System, Austin, TX (US)

(72) Inventors: Chun-Li Zhang, Coppell, TX (US); Lei-Lei Wang, Dallas, TX (US); Yuhua Zou, Coppell, TX (US); Wenjiao Tai, Dallas, TX (US); Wenze Niu, Dallas, TX (US)

(73) Assignee: The Board of Regents of the University of Texas System, Austin, TX (US)

(21) Appl. No.: 18/050,687

(22) Filed: Oct. 28, 2022

Related U.S. Application Data
(60) Provisional application No. 63/275,237, filed on Nov. 3, 2021.

(51) Int. Cl. C12N 5/0797; C12N 5/0775; C12N 5/077; C12N 15/86

(52) U.S. Cl. CPC C12N 5/0623; C12N 5/0662; C12N 5/0657; C12N 15/86; C12N 2501/10

(57) ABSTRACT

The present disclosure describes the use of ectopic SOX2 to promote nerve growth and regeneration, particularly in the context of nerve deficit stemming from trauma and disease, by reprogramming non-nerve cells into neuronal cells. In particular, the disclosure provides for the use of SOX2 therapy, optionally combined with neurogenic growth factors, to treat nerve deficit conditions.

### PRIORITY CLAIM

This application claims benefit of priority to U.S. Provisional Application Serial No. 63/275,237, filed Nov. 3, 2022, the entire contents of which are hereby incorporated by reference.

### STATEMENT REGARDING FEDERALLY FUNDED RESEARCH

This invention was made with government support under DP2 OD006484-01 awarded by the National Institutes of Health. The government has certain rights in the invention.

### INCORPORATION OF SEQUENCE LISTING

The sequence listing that is contained in the file named “UTFD-P2440US Sequence Listing.xml”, which is 7 KB (as measured in Microsoft Windows®) and was created on Oct. 27, 2022, is filed herewith by electronic submission and is incorporated by reference herein.

### BACKGROUND

### 1. Field

The present disclosure relates generally to the fields of medicine and neurobiology. More particularly, it concerns compositions and methods for the treatment of nerve deficits and nerve damage. Specifically, it relates to the use of elevated SOX2 expression to stimulate nerve regeneration and repair.

### 2. Description of Related Art

Neurogenesis persists in the adult mammalian brain and is modulated by many neurological conditions including injuries (Lie et al., 2004; Obernier et al., 2018). In contrast, new neurons are not normally detectable in the adult mammalian spinal cord (Horner et al., 2000). In response to spinal cord injury (SCI), several cell types including astrocytes and NG2 glia can proliferate; however, none of them are convincingly shown to generate mature neurons in vivo (Horky et al., 2006; Kang et al., 2010; Tripathi et al., 2010; Yamamoto et al., 2001). The central canal-lining ependymal cells, when isolated and cultured in vitro, can be induced to show stem cell-like properties giving rise to neurons, astrocytes, and oligodendrocytes (Meletis et al., 2008); nonetheless, they do not exhibit any stem cell function and fail to contribute to new cells in vivo after injury (Barnabe-Heider et al., 2010; Muthusamy et al., 2018; Ren et al., 2017; Shah et al., 2018). Thus, there remains a critical need to provide improved methods of stimulating the native cells that are capable of nerve self-repair.

### SUMMARY

Thus, in accordance with the present disclosure, there is provided a method of reprogramming a non-nerve cell into a neuronal cell comprising contacting said non-nerve cell with a SOX2 agonist. Also provided is a method of inducing regeneration, repair and/or growth of nerve tissue comprising contacting said a non-nerve cell in nerve tissue with a SOX2 agonist. The non-nerve cell may a glial cell or fibroblast and/or neuronal cell is a neuron. The neuron may be a brain neuron or a spinal neuron, or may be an inhibitory neuron or a excitatory neuron.

The SOX2 agonist may be a SOX2 protein or an expression construct comprising a SOX2 coding region under the control of a promoter active in mammalian cells. The promoter may be a tissue specific promoter, such as glial cell or stromal cell specific promoter, including but not limited to NG2, GFAP, or PDGFRA. The promoter may be a constitutive promoter or an inducible promoter. The expression construct may be a viral or non-viral expression vector.

The method may further comprise contacting said non-nerve cell with a neuronal growth factor, such as brain-derived neurotrophic factor (BDNF), noggin (NOG), or NT3. The non-nerve cell may be located in a living mammalian subject, such as a human. Alternatively, the non-nerve cell may be located outside of a living mammalian subject, such as where the non-nerve cell has been obtained from the subject, is contacted SOX agonist and optional a neuronal growth factor ex vivo, and then is returned a nerve site, such as a nerve injury or disease site, in said subject.

In embodiments, the subject has suffered a nerve injury, such as a spinal cord injury, traumatic brain injury, a stroke, or a neurodegenerative disease. The SOX2 agonist and/or the neuronal growth factor may be contacted with said non-nerve cell more than once. The method may further comprise treating said subject with physical therapy or other nerve deficit therapy prior to, at the time of, or post-contacting.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the disclosure, and vice versa. Furthermore, compositions and kits of the disclosure can be used to achieve methods of the disclosure.

Throughout this application, the term “about” is used to indicate that a value includes the inherent variation of error for the device, the method being employed to determine the value, or the variation that exists among the study subjects.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a device or a method that “comprises,” “has,” “contains,” or “includes” one or more elements possesses those one or more elements but is not limited to possessing only those one or more elements or steps. Likewise, an element of a device or method that “comprises,” “has,” “contains,” or “includes” one or more features possesses those one or more features but is not limited to possessing only those one or more features.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-L. SCI-induced neurogenic reprogramming of NG2 glia. (FIG. 1A) Experimental design for SCI-induced DCX⁺ cells. IHC, immunohistochemistry; wk, week. (FIG. 1B) Quantification of DCX⁺ cells induced by the indicated injury types (mean±SEM; n=3 mice per condition). n.d., not detected. (FIG. 1C) Confocal images of injury induced DCX⁺ cells at one-week post injury (wpi) in the adult mouse spinal cord. Enlarged views of the boxed regions are shown in the bottom panels. GFAP expression shows lesion areas. Nuclei are counterstained with Hoechst 33342 (Hst). Scale bars, 50 μm. (FIG. 1D) Experimental design for time-course analysis of SCI-induced DCX⁺ cells. (FIG. 1E) Quantification of crush-induced DCX⁺ cells at the indicated time-points (mean±SEM; n=4-5 mice per time-point). (FIG. 1F) Confocal images of crush-induced DCX⁺ cells through a time-course. Scale bars, 50 μm. (FIG. 1G) Experimental design for genetic lineage tracings of SCI-induced DCX⁺ cells. Tam, tamoxifen. (FIG. 1H) Quantification of genetically traced DCX⁺ cells at 1 wpi (mean±SEM; n=3-7 mice per group). (FIG. 1I) Lineage tracings showing SCI-induced DCX⁺ cells rarely originate from ependymal cells. An enlarged view of the boxed region is shown in the bottom panel. tdT is pseudocolored as green. Scale bars, 50 μm. (FIG. 1J) Lineage tracings showing SCI-induced DCX⁺ cells do not come from NES⁺ cells located in the central canal. An enlarged view of the boxed region is shown in the bottom panel. Scale bars, 50 μm. (FIG. 1K) lineage tracings showing SCI-induced DCX⁺ cells do not originate from resident astrocytes. An enlarged view of the boxed region is shown in the bottom panel. tdT is pseudocolored as green. Scale bars, 50 μm. (FIG. 1L) lineage tracings showing SCI-induced DCX⁺ cells largely originate from NG2 glia. An enlarged view of the boxed region is shown in the bottom panel. Scale bars, 50 μm. (M) Percentage of SCI-induced DCX⁺ cells expressing the indicated markers (mean±SEM; n=3 mice per group). See also FIGS. 10A-F.

FIGS. 2A-P. Cell-autonomous requirement of SOX2 for SCI-induced reprogramming of NG2 glia. (FIG. 2A) Experimental design for analyzing SOX2 expression in SCI-induced DCX⁺ cells. (FIG. 2B) Confocal images showing SOX2 expression in SCI-induced DCX⁺ cells. Scale bars, 50 μm. (FIG. 2C) Quantification of relative SOX2 expression (mean±SEM; n=6 mice per group; ****p<0.0001 by t-test). (FIG. 2D) Percentage of DCX⁺ cells with SOX2 co-expression (mean±SEM; n=6 mice per group; ****p<0.0001 by t-test). (FIG. 2E) Experimental design for inducible deletion of Sox2 in NG2 glia (NG2-cKO). Het, heterozygotes. (FIG. 2F) Confocal images showing inducible deletion of SOX2. Scale bars, 50 μm. (FIG. 2G) Quantification of Sox2-deleted NG2 glia (mean±SEM; n=4 mice per group; **p=0.0039 by t-test). (FIG. 2H) Experimental design for inducible deletion of Sox2 in NG2 glia. (FIG. 2I) Confocal images showing SCI-induced DCX⁺ cells in the indicated mouse spinal cord. Scale bars, 50 μm. (FIG. 2J) Quantification of SCI-induced DCX⁺ cells in the indicated mouse spinal cords (mean±SEM; n=3 mice per group; *p=0.0151 by t-test). (FIG. 2K) Experimental design for inducible deletion of Sox2 in astrocytes (Ast-cKO). (FIG. 2L) Confocal images showing inducible deletion of Sox2 in astrocytes. Scale bars, 50 μm. (FIG. 2M) Quantification of Sox2-deleted astrocytes (mean±SEM; n=6 mice per group; **p=0.0033 by t-test). (FIG. 2N) Experimental design for inducible deletion of Sox2 in astrocytes. (FIG. 2O) Confocal images showing SCI-induced DCX⁺ cells in the indicate mouse spinal cord. Scale bars, 50 μm. (FIG. 2P) Quantification of SCI-induced DCX⁺ cells in the indicated mouse spinal cords (mean±SEM; n=6 mice per group; n.s., not significant). See also FIG. S2.

FIGS. 3A-N. Elevated SOX2 is sufficient to drive neurogenic reprogramming of NG2 glia. (FIG. 3A) Experimental design for analyzing SOX2-induced DCX⁺ cells in NG2 glia. (FIG. 3B) Quantification of SOX2-induced DCX⁺ cells at 4 wpv (mean±SEM; n=5 mice per group; ***p=0.0008 by t-test; wpv: weeks post virus-injections). (FIG. 3C) Confocal images showing DCX⁺ cells induced by ectopic SOX2 in NG2 glia at 4 wpv. Scale bars, 50 μm. (FIG. 3D) Experimental design to genetically trace NG2 glia-derived cells. (FIG. 3E) Percentage of genetically traced cells converted into DCX⁺ cells at 4 wpv around the injection area (mean±SEM; n=4 mice per group; ***p=0.0003 by t-test). (FIG. 3F) Confocal images showing an origin of NG2 glia of SOX2-induced DCX⁺ cells. An orthogonal view of the boxed region is shown on the right. Scale bar, 50 μm. (FIG. 3G) Experimental design to genetically trace derivatives of ependymal cells. (FIG. 3H) Confocal images showing a non-ependymal cell origin of SOX2-induced DCX⁺ cells. An enlarged view of the boxed region is shown on the right. Scale bars, 50 μm. (FIG. 3I) Percentage of SOX2-induced DCX⁺ cells from genetically traced ependymal cells at 4 wpv (n=3-7 mice per group). (FIG. 3J) Experimental design to genetically trace derivatives of cells lining the central canal. (FIG. 3K) Confocal images showing a non-central canal cell origin of SOX2-induced DCX⁺ cells. An enlarged view of the boxed region is shown on the right. Scale bars, 50 μm. (FIG. 3L) Percentage of SOX2-induced DCX⁺ cells from genetically traced central canal cells at 4 wpv (n=3-4 mice per group). (FIG. 3M) Confocal images showing reprogramming efficiency by SOX2 in NG2 glia. Scale bars, 50 μm. (FIG. 3N) Quantification of reprogramming efficiency at 4 wpv (mean±SEM; n=5 mice per group). See also FIGS. 12.A-K

FIGS. 4A-U. Ectopic SOX2 reprograms NG2 glia into mature neurons. (FIG. 4A) Experimental design to determine proliferation of SOX2-induced DCX⁺ cells. (FIG. 4B) Percentage of SOX2-induced DCX⁺ cells going through proliferation (mean±SEM; n=5 mice per group). (FIG. 4C) Confocal images showing proliferation of SOX2-induced DCX⁺ cells at 4 wpv. Scale bars, 50 μm. (FIG. 4D) Experimental design for analyzing ASCL1⁺ progenitors. (FIG. 4E) Quantification of virus-induced ASCL1⁺ progenitors at 3 wpv (mean±SEM; n=3 mice per group; **p=0.0036 by t-test). (FIG. 4F) Confocal images showing SOX2-induced ASCL1⁺ progenitors at 3 wpv. Scale bars, 50 μm. (FIG. 4G) Experimental design to determine the NG2 glia origin for SOX2-induced ASCL1⁺ cells. (FIG. 4H) Quantification of SOX2-induced ASCL1⁺ progenitors from NG2 glia at 3 wpv (mean±SEM; n=3 mice per group; ****p<0.0001 by t-test). (FIG. 4I) Confocal images showing SOX2-induced ASCL1⁺ progenitors and DCX⁺ cells originating from NG2 glia at 3 wpv. Scale bars, 50 μm. (FIG. 4J) Experimental design to determine the lineage relationship of SOX2-induced ASCL1⁺ progenitors and DCX⁺ cells. (FIG. 4K) Quantification of DCX⁺ cells from SOX2-induced ASCL1⁺ progenitors at 4 wpv (mean±SEM; n=3 mice per group; **p=0.0015 by t-test). (FIG. 4L) Confocal images showing lineage-traced DCX⁺ cells from SOX2-induced ASCL1⁺ progenitors at 4 wpv. Scale bars, 50 μm. (FIG. 4M) Experimental design for analyzing SOX2-induced new neurons. (FIG. 4N) Quantification of SOX2-induced new neurons from NG2 glia (mean±SEM; n=3-6 mice per group; *p=0.0162 and ***p=0.0006 by t-test). (FIG. 4O) Confocal images of SOX2-induced new neurons from NG2 glia. Enlarged views of the boxed regions are shown in the right panels. Scale bars, 50 μm. (FIG. 4P) Experimental design for analyzing SOX2-induced neurons from NG2 glia. (FIG. 4Q) Confocal images of NG2 glia-derived neurons expressing markers for mature neurons. Examples of these neurons are indicated by arrows. Some of the YFP⁻NeuN⁻MAP2⁻ cells are outlined with dotted circles. Scale bars, μm. (FIGS. 4R-T) Confocal images of NG2 glia-derived neurons with the indicated subtype-specific markers. Asterisks indicate examples of NG2 glia-derived neurons, whereas arrowheads point to signal co-localization in orthogonal views. Some of the YFP⁻GLYT2⁻MAP2⁻ cells are outlined with dotted circles. Scale bars, 20 μm. (FIG. 4U) Quantification of subtypes of NG2 glia-derived neurons (mean±SEM; n=100-500 YFP⁺MAP2⁺ cells from 3 mice for each maker). See also FIGS. 13A-T and FIGS. 14A-E.

FIGS. 5A-K. SOX2-induced neurons from NG2 glia form synaptic connections. (FIG. 5A) Experimental design for analyzing monosynaptic connections of NG2 glia-derived neurons. (FIG. 5B) Confocal images of cells surrounding the virus-injected spinal cord area. Induced neurons are traced with tdT, whereas cells harboring the engineered rabies virus are indicated by eGFP. Arrows show examples of “starter” cells (eGFP⁺tdT⁺NeuN⁺). Some of the eGFP⁺tdT⁺NeuN⁺ cells are outlined with dotted circles. Scale bar, 20 μm. (FIG. 5C) Estimates of “starter” cells in the virus-injected spinal cord (mean±SEM; n=3-6 mice per group). (FIG. 5D) Confocal images of rabies-traced propriospinal neurons. Scale bars, 50 μm. (FIG. 5E) Estimates of rabies-traced propriospinal neurons (mean±SEM; n=3-6 mice per group). (FIG. 5F) Confocal images of rabies-traced DRG neurons. Scale bars, 200 μm. (FIG. 5G) Estimates of rabies-traced DRG neurons (mean±SEM; n=3-6 mice per group). (FIG. 5H) A schematic representation of spinal areas with induced neurons and rabies-traced neurons. (FIG. 5I) Confocal images of rabies-traced axon bundles at the indicated spinal cord levels. Their approximate locations are marked in the top diagrams. G/C, gracile/cuneate tract; ReST, reticulospinal tract; VeST, vestibulospinal tract. Scale bars, 100 μm. (FIG. 5J) Schematic representations and confocal images of rabies-traced neurons in the brainstem. Rabies-traced axon bundles and somas are indicated in the top diagrams. RS, rubrospinal tract; RetN, reticular nucleus; VN, vestibular nucleus; RF, reticular formation. Scale bars, 20 μm. (FIG. 5K) Estimates of rabies-traced neurons in the brain/brainstem (mean±SEM; n=3-6 mice per group). See also FIGS. 15A-E.

FIGS. 6A-K. SOX2-mediated reprogramming of NG2 glia promotes functional recovery following SCI. (FIG. 6A) A schematic drawing of the procedure. Adult mouse underwent dorsal hemisection at the 5^{th} cervical level (C5-DH), followed by virus injections and behavioral tests. (FIG. 6B) Experimental design for behavioral tests. (FIG. 6C) Percentage of forelimb drops during grid-walking tests (mean±SEM; n=12-13 mice per group; F(14,454)=176.5 and p<0.0001 for time-dependent effect; F(2,35)=4.299 and p=0.0214 for treatment-dependent effect; SOX2/p75-2 vs. GFP: ^{##}p=0.0015 at 14 wks, ^{#}p=0.0140 at 16 wks, ^{##}p=0.0013 at 18 wks, ^{#}p=0.02223 at 20 wks, ^{####}p<0.0001 at 22 wks, 24 wks and 26 wks; SOX2/p75-2 vs. p75-2: *p=0.0261 at 14 wks, *v0.0346 at 22 wks, *p=0.0488 at 24 wks, and *p=0.0383 at 26 wks). The inset graph shows performance of individual mouse at the endpoint 26 wks. (FIG. 6D) Lower magnification views of spinal cord tissues with the indicated marker staining. Scale bars, 200 μm. (FIG. 6E) Enlarged views of the boxed regions in FIG. 6D. Arrowheads show examples of BrdU⁺NeuN⁺ neurons. Higher magnification views of the boxed regions are shown in the lower panels. Scale bars, 25 μm. (FIG. 6F) A schematic of the distribution of SOX2-induced new neurons. D, dorsal; V, ventral; R, rostral; C, caudal. (FIG. 6G) Quantification of new neurons in the injured spinal cord (mean±SEM; n=6 mice per group). (FIG. 6H) Low (stitched images) and high magnification views of GFAP-stained glial scar surrounding the lesion epicenter. Scale bars, 500 μm and 50 μm for the left and right panels, respectively. (FIG. 6I) Representative 3D reconstructions of glial scar at the lesion site. Scale bars, 500 μm. (FIG. 6J) Quantification of glial scar volume (mean±SEM; n=8 mice per group; *p=0.0247). (FIG. 6K) Quantification of glial scar surface area (mean±SEM; n=8 mice per group; *p=0.0361). See also FIGS. 16A-B.

FIGS. 7A-H. SOX2-reprogrammed NG2 glia are bipotent in the spinal cord with injury. (FIG. 7A) A schematic drawing of C5-DH SCI and the locations of virus injections. (FIG. 7B) Experimental design for analyzing the fates of SOX2-reprogrammed NG2 glia in adult Ascl1-CreER^{T2};Rosa-tdT mice. (FIG. 7C) Stitched images of lower magnification views of spinal cords injected with the indicated viruses at 12 wpv. Scale bar, 250 μm. (FIGS. 7D-G) Confocal images of the indicated cell markers surrounding the injured spinal cord. Scale bars, 50 μm. (FIG. 7H) Quantification of the fates of lineage traced cells (mean±SEM; n=3-4 mice per group).

FIG. 8. Examplary mRNA sequences for human and mouse SOX-2.

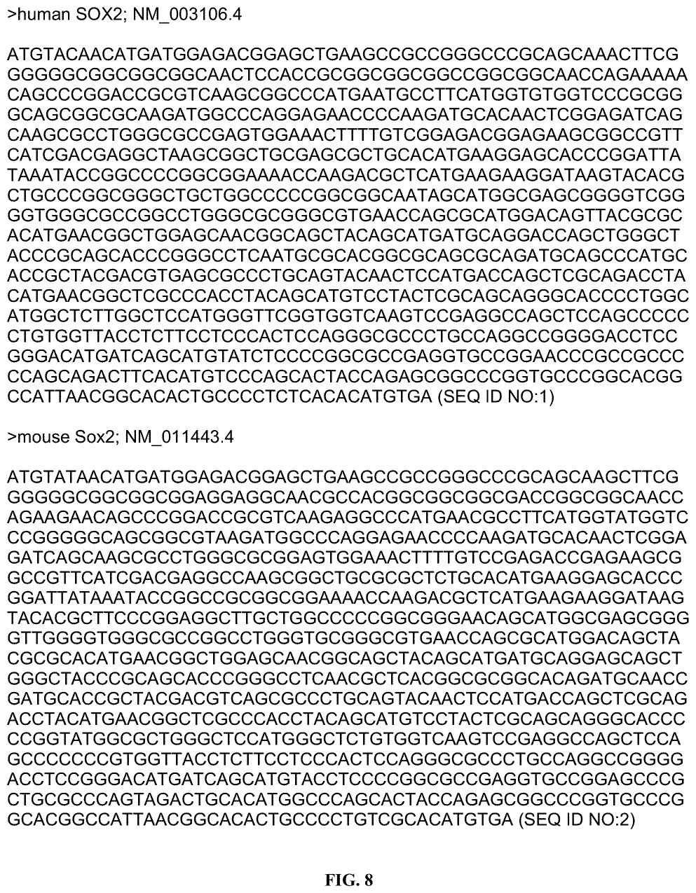

FIG. 9. Examplary protein sequences for human and mouse SOX-2.

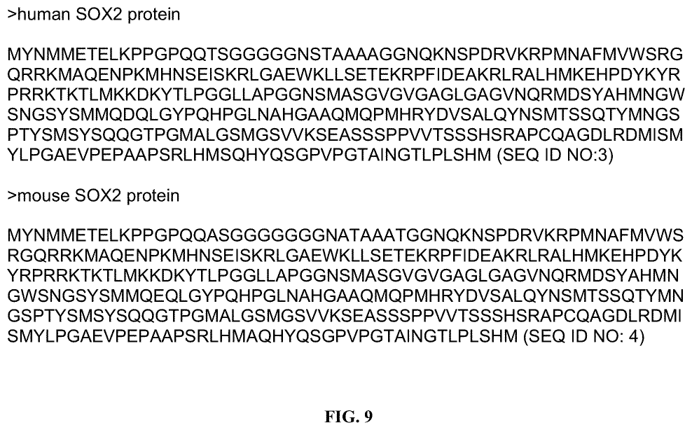

FIGS. 10A-F. Related to FIGS. 1A-M. Genetic lineage-tracing and additional marker expression of SCI-induced DCX⁺ cells. (FIG. 10A) Tracing ependymal cells lining the central canal in adult Foxj1-CreER^{T2};Rosa-tdT mice. tdT is pseudocolored as green. Scale bar, 50 μm. (FIG. 10B) Tracing spinal neural stem cells lining the central canal in adult Nes-CreER^{T2};Rosa-YFP mice. Scale bar, 50 μm. (FIG. 10C) Robust labeling of neural stem cells and DCX⁺ cells in the lateral ventricle of adult Nes-CreER^{T2};Rosa-YFP mice. Scale bar, 50 μm. (FIGS. 10D-F) Confocal images of the indicated markers surrounding the injured spinal cord regions. Mice with crush injury were analyzed at 1-week post injury (wpi). Enlarged views of the boxed regions are shown on the bottom panels. Scale bars, 50 μm.

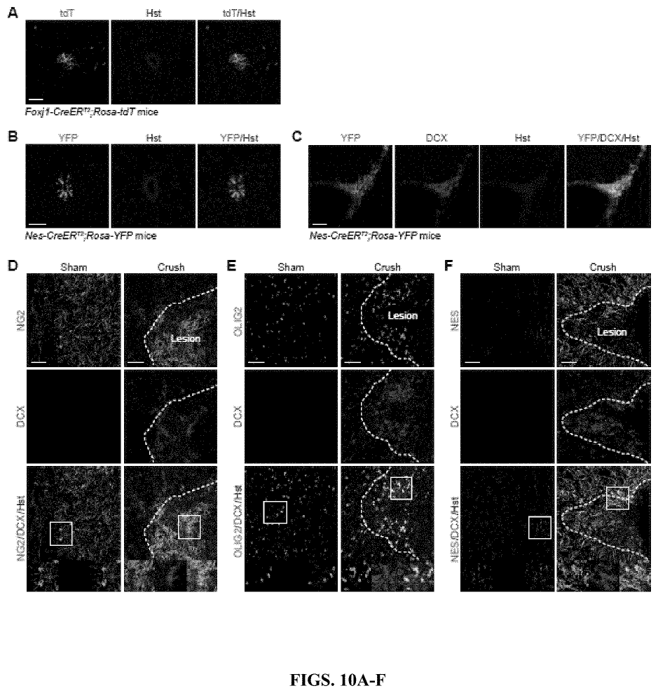

FIG. 11. Related to FIGS. 2A-P. Predominant macroglial expression of SOX2 in the injured spinal cord. Confocal images of the indicated markers surrounding the contusion-injured spinal cord. Immunostaining were performed at 1 wpi. Enlarged views of the boxed regions are shown on the bottom panels. Scale bars, 50 μm.

FIGS. 12A-K. Related to FIGS. 3A-N. The specificity of hNG2 promoter and genetic tracing of NG2 glia and their derivatives. (FIG. 12A) Experimental design for examining the specificity of hNG2 promoter. (FIG. 12B) Quantification of marker expression in GFP⁺ cells (mean±SEM; n=3 mice). At least 500 GFP⁺ cells were analyzed for each marker. (FIG. 13C) A stitched image of lower magnification view of a longitudinal section from the adult spinal cord injected with the hNG2-GFP lentivirus at one-week post-virus-injection (wpv). Arrows show the viral injection sites. Scale bar, 250 μm. (FIG. 12D) Confocal images of GFP⁺ cells in the adult spinal cord. Enlarged views of the boxed regions are shown on the right panels. Scale bars, 50 μm. (FIG. 12E) Experimental design for genetic lineage tracing of NG2 glia. (FIG. 12F) Quantification of marker expression in YFP⁺ cells (mean±SEM; n=3 mice). At least 100 YFP⁺ cells were analyzed for each marker. n.d., not detected. (FIG. 12G) Confocal images of the indicated markers in the spinal cord of adult Pdgfra-CreER^{TM};Rosa-YFP mice 2 weeks post tamoxifen administration. Enlarged views of the boxed regions are shown on the right panels. Scale bars, 50 μm. (FIG. 12H) A schematic for restricted SOX2 expression in NG2 glia of Pdgfra-CreER^{TM};Rosa-tdT mice. (FIG. 12I) Experimental design. (FIG. 12J) Quantification of SOX2-induced DCX⁺ cells from NG2 glia (mean±SEM; n=3 mice). n.d., not detected. (K) Confocal images of the indicated markers in the spinal cord of adult Pdgfra-CreER^{TM};Rosa-tdT mice. Scale bars, 20 μm.

FIGS. 13A-T. Related to FIGS. 4A-U. SCI-reprogrammed NG2 glia fail to generate mature neurons. (FIG. 13A) Experimental design for SCI-induced cell proliferation. (FIGS. 13B-C) Quantification and confocal images of SCI-induced DCX⁺ cells going through proliferation (mean±SEM; n=3 mice per group). Scale bars, 50 μm. (FIG. 13D) Confocal images of the indicated marker expression at 1 wpi. Enlarged views of the boxed regions are shown on the right panels. Scale bars, 50 μm. (FIG. 13E) Percentage of DCX⁺ cells expressing ASCL1 (mean±SEM; n=3 mice per group). (FIG. 13F) Experimental design for assessing the fate of SCI-induced ASCL1⁺ cells. (FIGS. 13G-K) Confocal images of the indicated markers in the spinal cord of adult Ascii-CreER^{T2};Rosa-tdT mice at 4 wpi. Scale bars, 50 μm. (FIG. 13L) Quantification of marker expression in tdT⁺ cells (mean±SEM; n=3 mice). (FIG. 13M) Experimental design for analyzing SCI-induced new neurons. (FIG. 13N) Quantification of SCI-induced new neurons (mean±SEM; n=3 mice per group). (FIG. 13O) Confocal images of SCI-induced new neurons at 8 wpi. An orthogonal view of the boxed region is also shown in the bottom panel. Scale bars, 50 μm. (FIG. 13P) Experimental design for investigating the effect of lentivirus injection on SCI-induced DCX⁺ cells. (FIG. 13Q) Quantification of DCX⁺ cells (mean±SEM; n=3-4 mice per group). (FIG. 13R) Confocal images of DCX⁺ cells in the spinal cord one-week post SCI and virus injection. Scale bars, 50 μm. (FIG. 13S) Experimental design for analyzing the effect of neurotrophic factors on SCI-induced neurons. (FIG. 13T) Quantification of the effect of neurotrophic factors on SCI-induced new neurons (mean±SEM; n=3 mice per group).

FIGS. 14A-E. Related to FIGS. 4A-U. Additional analysis of SOX2-mediated reprogramming of NG2 glia. (FIG. 14A) Experimental design for BrdU-tracing. (FIG. 14B) Confocal images of the indicated markers at 8 wpv, a time point with many BrdU⁺NeuN⁺ neurons but few DCX⁺ cells. Scale bars, 50 μm. (FIG. 14C) Experimental design for analyzing SOX2-induced neurons from NG2 glia. (FIG. 14D) Expression of the presynaptic marker SYN1 in induced neurons. Asterisks indicate examples of NG2 glia-derived neurons, whereas arrowheads show signal co-localization in the orthogonal view. Scale bar, 20 μm. (FIG. 14E) Expression of the inhibitory neuronal marker VGAT in induced neurons. Asterisk indicates an example of NG2 glia-derived neurons, whereas arrowheads show signal co-localization in the orthogonal view. Scale bar, 20 μm.

FIGS. 15A-E. Related to FIGS. 5A-K. Rabies virus-mediated tracing of synaptic connections of NG2 glia-derived neurons. (FIG. 15A) Confocal images of cells in the spinal cord (SC) and dorsal root ganglia (DRG) of the control Ascl1-CreER^{T2};Rosa-tdT mice that were injected with SOX2/p75-2 virus and the engineered rabies virus. Induced tdT⁺NeuN⁺ neurons were only observed in the SC and were not infected with the engineered rabies virus due to the lack of TVA expression (blue arrowheads for examples; n=4). WM, white matter. Scale bars, 100 μm. (FIG. 15B) Confocal images of cells in the SC and DRG of Ascl1-CreER^{T2};Rosa-tdT;Rosa-TVAg mice that were injected with the control p75-2 virus and the engineered rabies virus. tdT⁺NeuN⁺ neurons were not detected. Only rare glial cells were infected with the engineered rabies virus (white arrowheads and enlarged views). GM, gray matter. Scale bars, 100 μm. (FIG. 15C) Stitched images of lower magnification views of the spinal cord and the attached DRGs (outlined) of Ascl1-CreER^{T2};Rosa-tdT;Rosa-TVAg mice that were injected with SOX2/p75-2 virus and the engineered rabies virus. The relative positions of the longitudinal sections are diagramed on the top panels. Scale bar, 1 mm. (FIG. 15D) Confocal images of cells from the ventral white matter surrounding the virus-injected spinal cord. Induced neurons are traced with tdT, whereas cells harboring the engineered rabies virus are indicated by eGFP. Asterisks show an example of an eGFP⁺tdT⁺NeuN⁺ “starter” cell. Scale bar, 50 μm. (FIG. 15E) Confocal images of clustered “starter” cells in the virus-injected spinal cord. Both maximum projections (left, scale bar, 100 μm) and a series of single confocal planes (right, scale bars, 20 μm) are shown.

FIGS. 16A-B. Related to FIGS. 6A-K. 3D reconstructed astroglial scar. (FIG. 16A) Contouring of the astroglial scar. Left, a contour line in a representative GFAP-labeled astroglial scar in a representative stitched image (scale bar, 500 μm); Right, Neurolucida 3D reconstruction of contours of the astroglial scar. (FIG. 16B) 3D reconstructed astroglial scar from each case in all groups.

### DESCRIPTION OF ILLUSTRATIVE EMBODIMENTS

As discussed above, nerve deficits and injuries constitute a major challenge for health care providers and represent a tremendous financial strain on insurance companies as well as individuals suffering from such injuries. One significant example is spinal cord injury (SCI), which commonly results in permanent paralysis and sensory impairments due to poor spontaneous nerve regeneration in the central nervous system. This is even more exacerbated when the injury results in tissue loss as a consequence of trauma. This normally follows by cell death at the injury epicenter, which forms a fluid filled cyst that prevents axonal regeneration.

Another significant example is nerve injury in the upper and lower extremities, which also results in permanent paralysis and sensory impairments due to poor spontaneous nerve regeneration in the peripheral nervous system. Even when a nerve is cut and directly repaired, the recovery is suboptimal and represents only a fraction of the pre-injury function. This is even more exacerbated when the injury results in tissue loss as a consequence of trauma. The nerve has no potential for spontaneous regeneration across this nerve tissue defect, or nerve gap.

In this study, the inventors reexamined cellular responses to SCI by expression of the microtubule-associated protein doublecortin (DCX). DCX is normally expressed in neuroblasts and immature neurons and can serve as a reliable marker for adult neurogenesis (Couillard-Despres et al., 2005; Kempermann et al., 2004; Knoth et al., 2010). It is highly expressed in the developing spinal cord but completely turned off in the adult (Juhasova et al., 2015). Although DCX⁺ cells were previously observed surrounding the lesion site in the adult spinal cord (Ziv et al., 2006), their origin and final fate is not clear. Genetic lineage mappings show that SCI induces transient DCX expression in NG2 glia but not astrocytes or ependymal cells. However, these injury-induced phenotypically reprogrammed NG2 glia fail to produce new neurons even in the presence of exogenous neurotrophic factors. The inventors further show that SOX2 is both required and sufficient in a cell-autonomous manner for neurogenic reprogramming of NG2 glia. Ectopic SOX2 also enables these NG2 glia to generate propriospinal neurons that make synaptic connections with endogenous neurons located in the brain and spinal cord. Moreover, SOX2-mediated reprogramming of NG2 glia contributes to behavioral improvements after cervical SCI. These data indicate that injury-induced transient neurogenic reprogramming of NG2 glia can be enhanced for neural regeneration.

These and other aspects of the disclosure are described in detail below.

### 1. NERVE INJURIES AND DEFECTS

Nerve injury, broadly defined, is injury to nervous tissue. There is no single classification system that can describe all the many variations of nerve injury. In 1941, Seddon introduced a classification of nerve injuries based on three main types of nerve fiber injury and whether there is continuity of the nerve. Usually, however, (peripheral) nerve injury is classified in five stages, based on the extent of damage to both the nerve and the surrounding connective tissue, since supporting glial cells may be involved. Unlike in the central nervous system, neuroregeneration in the peripheral nervous system is possible. The processes that occur in peripheral regeneration can be divided into the following major events: Wallerian degeneration, axon regeneration/growth, and end-organ reinnervation. The events that occur in peripheral regeneration occur with respect to the axis of the nerve injury. The proximal stump refers to the end of the injured neuron that is still attached to the neuron cell body; it is the part that regenerates. The distal stump refers to the end of the injured neuron that is still attached to the end of the axon; it is the part of the neuron that will degenerate but that remains in the area toward which the regenerating axon grows.

Neuropraxia is the least severe form of nerve injury, with complete recovery. In this case, the axon remains intact, but there is myelin damage causing an interruption in conduction of the impulse down the nerve fiber. Most commonly, this involves compression of the nerve or disruption to the blood supply (ischemia). There is a temporary loss of function which is reversible within hours to months of the injury (the average is 6-9 weeks). Wallerian degeneration does not occur, so recovery does not involve actual regeneration. In electrodiagnostic testing with nerve conduction studies, there is a normal compound motor action potential amplitude distal to the lesion at day 10, and this indicates a diagnosis of mild neuropraxia instead of axonotmesis or neurotmesis.

Axonotmesis a more severe nerve injury with disruption of the neuronal axon, but with maintenance of the epineurium. This type of nerve damage may cause loss of the motor, sensory, and autonomic functions. This is mainly seen in crush injury.

If the force creating the nerve damage is removed in a timely fashion and the surrounding tissue is preserved, the axon may regenerate, leading to recovery. Electrically, the nerve shows rapid and complete degeneration, with loss of voluntary motor units. Regeneration of the motor end plates will occur, as long as the endoneural tubules are intact.

Axonotmesis involves the interruption of the axon and its covering of myelin but preservation of the connective tissue framework of the nerve (the encapsulating tissue, the epineurium and perineurium, are preserved). Because axonal continuity is lost, Wallerian degeneration occurs. Electromyography (EMG) performed 2 to 4 weeks later shows fibrillations and denervation potentials in musculature distal to the injury site. Loss in both motor and sensory spines is more complete with axonotmesis than with neurapraxia, and recovery occurs only through regenerations of the axons, a process requiring time.

Axonotmesis is usually the result of a more severe crush or contusion than neuropraxia but can also occur when the nerve is stretched (without damage to the epineurium). There is usually an element of retrograde proximal degeneration of the axon, and for regeneration to occur, this loss must first be overcome. The regenerating fibers must cross the injury site and regeneration through the proximal or retrograde area of degeneration may require several weeks. Then the neurite tips progress down to the distal site, such as the wrist or hand. Proximal lesion may grow distally as fast as 2 to 3 mm per day and distal lesion as slowly as 1.5 mm per day. Regeneration occurs over weeks to years.

Neurotmesis is the most severe lesion with no potential of full recovery. It occurs on severe contusion, stretch, laceration, or local anesthetic toxicity. The axon and encapsulating connective tissue lose their continuity. The last (extreme) degree of neurotmesis is transection, but most neurotmetic injuries do not produce gross loss of continuity of the nerve but rather internal disruption of the architecture of the nerve sufficient to involve perineurium and endoneurium as well as axons and their covering. Denervation changes recorded by EMG are the same as those seen with axonotmetic injury. There is a complete loss of motor, sensory and autonomic function. If the nerve has been completely divided, axonal regeneration causes a neuroma to form in the proximal stump. For neurotmesis, it is better to use a new more complete classification called the Sunderland System.

The following are exemplary nerve defects that can be addressed by the approach outlined in the present disclosure.

A. Spinal Cord Nerve Defects

A spinal cord injury (SCI) or defect is an injury to the spinal cord resulting in a disruption, either temporary or permanent, in the cord's normal motor, sensory, or autonomic function. Common causes of damage are trauma (car accident, gunshot, falls, sports injuries, etc.) or disease (transverse myelitis, polio, spina bifida, Friedreich's ataxia, etc.). The spinal cord does not have to be severed in order for a loss of function to occur. Depending on where the spinal cord and nerve roots are damaged, the symptoms can vary widely, from pain to paralysis to incontinence. Spinal cord injuries are described at various levels of “incomplete,” which can vary from having no effect on the patient to a “complete” injury which means a total loss of function.

Treatment of spinal cord injuries starts with restraining the spine and controlling inflammation to prevent further damage. The actual treatment can vary widely depending on the location and extent of the injury. In many cases, spinal cord injuries require substantial physical therapy and rehabilitation, especially if the patient's injury interferes with activities of daily life.

Research into treatments for spinal cord injuries includes nerve regeneration through the use of nerve growth factors, controlled hypothermia and stem cells, though many treatments have not been studied thoroughly and very little new research has been implemented in standard care.

B. Brain Injury and Cranial Nerve Deficits

Brain damage or brain injury (BI) is the destruction or degeneration of brain cells, including nerves. Brain injuries occur due to a wide range of internal and external factors. A common category with the greatest number of injuries is traumatic brain injury (TBI) following physical trauma or head injury from an outside source, and the term acquired brain injury (ABI) is used in appropriate circles to differentiate brain injuries occurring after birth from injury due to a disorder or congenital malady.¹

In general, brain damage refers to significant, undiscriminating trauma-induced damage, while neurotoxicity typically refers to selective, chemically induced neuron damage. Brain injuries occur due to a very wide range of conditions, illnesses, injuries, and as a result of iatrogenesis (adverse effects of medical treatment). Possible causes of widespread brain damage include birth hypoxia, prolonged hypoxia (shortage of oxygen), poisoning by teratogens (including alcohol), infection, and neurological illness. Chemotherapy can cause brain damage to the neural stem cells and oligodendrocyte cells that produce myelin. Common causes of focal or localized brain damage are physical trauma (traumatic brain injury, stroke, aneurysm, surgery, other neurological disorder), and poisoning from heavy metals including mercury and compounds of lead.

Cranial nerve disease is an impaired functioning of one of the twelve cranial nerves. It is possible for a disorder of more than one cranial nerve to occur at the same time, if a trauma occurs at a location where many cranial nerves run together, such as the jugular fossa. A brainstem lesion could also cause impaired functioning of multiple cranial nerves, but this condition would likely also be accompanied by distal motor impairment.

The facial nerve is the seventh of 12 cranial nerves. This cranial nerve controls the muscles in the face. Facial nerve palsy is more abundant in older adults than in children and is said to affect 15-40 out of 100,000 people per year. This disease comes in many forms which include congenital, infectious, traumatic, neoplastic, or idiopathic. The most common cause of this cranial nerve damage is Bell's palsy (idiopathic facial palsy) which is a paralysis of the facial nerve. Although Bell's palsy is more prominent in adults it seems to be found in those younger than 20 or older than 60 years of age. Bell's palsy is thought to occur by an infection of the herpes virus which may cause demyelination and has been found in patients with facial nerve palsy. Symptoms include flattening of the forehead, sagging of the eyebrow, and difficulty closing the eye and the mouth on the side of the face that is affected. The inability to close the mouth causes problems in feeding and speech. It also causes lack of taste, lacrimation, and sialorrhea.

C. Peripheral Nerve Deficits

Peripheral nerve damage is categorized in the Seddon classification based on the extent of damage to both the nerve and the surrounding connective tissue since the nervous system is characterized by the dependence of neurons on their supporting glia. Unlike in the central nervous system, regeneration in the peripheral nervous system is possible. The processes that occur in peripheral regeneration can be divided into the following major events: Wallerian degeneration, axon regeneration/growth, and end-organ reinnervation. The events that occur in peripheral regeneration occur with respect to the axis of the nerve injury. The proximal stump refers to the end of the injured neuron that is still attached to the neuron cell body; it is the part that regenerates. The distal stump refers to the end of the injured neuron that is still attached to the end of the axon; it is the part that will degenerate but remains the area that the regenerating axon grows toward.

The lowest degree of nerve injury in which the nerve remains intact but its signaling ability is damaged is called neurapraxia. The second degree in which the axon is damaged, but the surrounding connecting tissue remains intact is called axonotmesis. The last degree in which both the axon and connective tissue are damaged is called neurotmesis.

### 2. NEURONAL REPROGRAMING FACTORS

A. SOX2

SRY (sex determining region Y)-box 2, also known as SOX2, is a transcription factor that is essential for maintaining self-renewal, or pluripotency, of undifferentiated embryonic stem cells. Sox2 has a critical role in maintenance of embryonic and neural stem cells. Sox2 is a member of the Sox family of transcription factors, which have been shown to play key roles in many stages of mammalian development. This protein family shares highly conserved DNA binding domains known as HMG (High-mobility group) box domains containing approximately 80 amino acids. Sox2 holds great promise in research involving induced pluripotency, an emerging and very promising field of regenerative medicine. A reference mRNA sequence can be found at NM_003106 and a reference protein can be found at NP 003097. SOX2 has been shown to interact with PAX6, NPM1, and Oct4. SOX2 has been found to cooperatively regulate Rex1 with Oct3/4.

LIF (Leukemia inhibitory factor) signaling, which maintains pluripotency in mouse embryonic stem cells, activates Sox2 downstream of the JAK-STAT signaling pathway and subsequent activation of Klf4 (a member of the family of Kruppel-like factors). Oct-4, Sox2 and Nanog positively regulate transcription of all pluripotency circuitry proteins in the LIF pathway.

NPM1, a transcriptional regulator involved in cell proliferation, individually forms complexes with Sox2, Oct4 and Nanog in embryonic stem cells. These three pluripotency factors contribute to a complex molecular network that regulates a number of genes controlling pluripotency. Sox2 binds to DNA cooperatively with Oct4 at non-palindromic sequences to activate transcription of key pluripotency factors. Surprisingly, regulation of Oct4-Sox2 enhancers can occur without Sox2, likely due to expression of other Sox proteins. However, a group of researchers concluded that the primary role of Sox2 in embryonic stem cells is controlling Oct4 expression, and they both perpetuate their own expression when expressed concurrently.

In an experiment involving mouse embryonic stem cells, it was discovered that Sox2 in conjunction with Oct4, c-Myc and Klf4 were sufficient for producing induced pluripotent stem cells. The discovery that expression of only four transcription factors was necessary to induce pluripotency allowed future regenerative medicine research to be conducted considering minor manipulations.

Loss of pluripotency is regulated by hypermethylation of some Sox2 and Oct4 binding sites in male germ cells and post-transcriptional suppression of Sox2 by miR134. Varying levels of Sox2 affect embryonic stem cells' fate of differentiation. Sox2 inhibits differentiation into the mesendoderm germ layer and promotes differentiation into neural ectoderm germ layer. Npm1/Sox2 complexes are sustained when differentiation is induced along the ectodermal lineage, emphasizing an important functional role for Sox2 in ectodermal differentiation. One study showed, through the development of a knockout model, that deficiency of Sox2 results in neural malformities and eventually fetal death, further underlining Sox2's vital role in embryonic development.

In neurogenesis, Sox2 is expressed throughout developing cells in the neural tube as well as in proliferating central nervous system progenitors. However, Sox2 is downregulated during progenitors' final cell cycle during differentiation when they become post mitotic. Cells expressing Sox2 are capable of both producing cells identical to themselves and differentiated neural cell types, two necessary hallmarks of stem cells. Thus signals controlling Sox2 expression in the presumptive neuronal compartment, like Notch signaling, control what size the neuronal compartment finally reaches. Proliferation of Sox2+ neural stem cells can generate neural precursors as well as Sox2+ neural stem cell population. Differences in brain size between the species thus relate to the capacity of different species to maintain SOX2 expression in the developing neural systems. The difference in brain size between humans and apes, for instance, has been linked to mutations in the gene Asb11, which is an upstream activator of SOX2 in the developing neural system.

Induced pluripotency is possible using adult neural stem cells, which express higher levels of Sox2 and c-Myc than embryonic stem cells. Therefore, only two exogenous factors, one of which is necessarily Oct4, are sufficient for inducing pluripotent cells from neural stem cells, lessening the complications and risks associated with introducing multiple factors to induce pluripotency.

B. Pharmaceutical Formulations and Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

The active compositions of the present invention may include classic pharmaceutical preparations. One will generally desire to employ appropriate salts and buffers to render agents stable and allow for uptake by target cells. Aqueous compositions of the present invention comprise an effective amount of the agent(s) to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the present invention, its use in therapeutic compositions is contemplated. Supplementary active ingredients also can be incorporated into the compositions.

Administration of these compositions according to the present invention will be via an appropriate route but are particularly drawn to administration local or regional to a nerve deficit. Administration may be by injection or infusion. Such compositions would normally be administered as pharmaceutically acceptable compositions. When the route is topical, the form may be a cream, ointment, or salve.

An effective amount of the therapeutic agent is determined based on the intended goal, i.e., improving nerve growth, reducing a nerve deficit, and/or bridging a “critical gap.” The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

As used herein, the term in vitro preparation refers to manipulations performed on materials outside of the living animal. The term ex vivo administration refers to materials that have been manipulated in vitro and are subsequently administered to a living animal. The term in vivo administration includes all manipulations performed within an animal. In certain aspects of the present invention, the compositions may be prepared in vitro or administered either ex vivo or in vivo.

In the case of surgical intervention, the present invention may be used preoperatively, during surgery, or post-operatively. The administration may be continued post-surgery, for example, by leaving a catheter implanted at the site of the surgery. Periodic post-surgical treatment also is envisioned. Generally, the dose of the therapeutic composition via continuous perfusion will be equivalent to that given by single or multiple injections, adjusted over a period of time during which the perfusion occurs.

Treatment regimens may vary as well, and often depend on deficit type, deficit location, and health and age of the patient. Obviously, certain types of deficits will require more aggressive treatment, while at the same time, certain patients cannot tolerate more taxing protocols. The clinician will be best suited to make such decisions based on the known efficacy and toxicity (if any) of the therapeutic formulations.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are dimethyl sulfoxide, propylene glycol, polyethylene glycol, vegetable oil and injectable organic esters such as ethyloleate. Aqueous carriers include water, alcoholic/aqueous solutions, saline solutions, parenteral vehicles such as sodium chloride or Ringer's dextrose. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

C. Delivery of SOX2

The methods described herein involve the contacting of non-nerve cells with SOX2 protein. This can be accomplished in two ways. First SOX2 protein or mRNA may be delivered or, second, SOX2 protein may be provided via expression from a SOX2 expression cassette. The following is a general discussion of each approach.

i. Protein or mRNA Delivery

In general, proteins or mRNA may be delivered to cells as a formulation that promotes entry of the proteins or mRNA into a cell of interest. In a most basic form, this can include lipid vehicles such as liposomes or, alternatively, nanoparticles. For example, liposomes, which are artificially prepared vesicles made of lipid bilayers have been used to delivery a variety of drugs. Liposomes can be composed of naturally-derived phospholipids with mixed lipid chains (like egg phosphatidylethanolamine) or other surfactants. In particular, liposomes containing cationic or neutural lipids have been used in the formulation of drugs. Liposomes should not be confused with micelles and reverse micelles composed of monolayers, which also can be used for delivery.

A wide variety of commercial formulations for protein or mRNA delivery are well known including PULSin™, Lipodin-Pro, Carry-MaxR, Pro-DeliveriN, PromoFectin, Pro-Ject, Chariot™ Protein Delivery reagent, BioPORTER™, and others.

Nanoparticles are generally considered to be particulate substances having a diameter of 100 nm or less. In contrast to liposomes, which are hollow, nanoparticles tend to be solid. Thus, the drug will be less entrapped and more either embedded in or coated on the nanoparticle. Nanoparticles can be made of metals including oxides, silica, polymers such as polymethyl methacrylate, and ceramics. Similarly, nanoshells are somewhat larger and encase the delivered substances with these same materials. Either nanoparticles or nanoshells permit sustained or controlled release of the peptide or mimetic and can stabilize it to the effects of in vivo environment.

ii. Expression Cassette Delivery

As discussed above, in certain embodiments, expression cassettes are employed to express a protein product. Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Regulatory Elements. Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard and Schaffner, 1984 |
| T-Cell Receptor | Luria et al., 1987; Winoto et al., 1989; Redondo et |
| | al.; 1990 |
| HLA DQ a and/or DQ ß | Sullivan et al., 1987 |
| ß-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| ß-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere and Tilghman et al., |
| | 1989 |
| t-Globin | Bodine et al., 1987; Perez-Stable et al., 1990 |
| ß-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr and Clarke, 1986; |
| | Imbra et al., 1986; Kadesch et al., 1986; Wang and |
| | Calame, 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | and Botchan, 1986; Cripe et al., 1987; Gloss et al., |
| | 1987; Hirochika et al., 1987; Stephens et al., 1987 |
| Hepatitis B Virus | Bulla et al., 1986; Jameel et al., 1986; Shaul et al., |
| | 1987; Spandau and Lee, 1988; Vannice et al., 1988 |
| Human Immunodeficiency Virus | Muesing et al., 1987; Hauber et al., 1988; |
| | Jakobovits et al., 1988; Feng and Holland., 1988; |
| | Takebe et al., 1988; Rosen et al., 1988; Berkhout et |
| | al., 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse mammary | Glucocorticoids | Huang et al., 1981; Lee et |
| tumor virus) | | al., 1981; Majors et al., |
| | | 1983; Chandler et al., |
| | | 1983; Ponta et al., 1985; |
| | | Sakai et al., 1988 |
| ß-Interferon | poly(rI)x | Tavernier et al., 1983 |
| | poly(rc) | |
| Adenovirus 5 E2 | EIA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | EIA, SV40 Large T Antigen | Taylor et al., 1989, 1990a, |
| | | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Of particular interest are glial cell or stromal cell specific promoters, including but not limited to NG2, GFAP, or PDGFRA.

Polyadenylation signals. Where a cDNA insert is employed, one will typically desire to include a polyadenylation signal to effect proper polyadenylation of the gene transcript. The nature of the polyadenylation signal is not believed to be crucial to the successful practice of the disclosure, and any such sequence may be employed such as human growth hormone and SV40 polyadenylation signals. Also contemplated as an element of the expression cassette is a terminator. These elements can serve to enhance message levels and to minimize read through from the cassette into other sequences.

Multicistronic elements. In certain embodiments of the disclosure, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

Delivery of Expression Vectors. There are a number of ways in which expression vectors may introduced into cells. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One particular method for in vivo delivery involves the use of an adeno-associated virus (AAV) expression vector. AAV can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

Another expression vector may comprise a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Other viral vectors may be employed as expression constructs in the present disclosure. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the disclosure, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the disclosure for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment of the disclosure, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000™ is widely used and commercially available.

In certain embodiments of the disclosure, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

D. Combination Treatments

The inventors have determined that combinations of the aforementioned agents are particularly efficacious in addressing nerve deficits and promoting nerve growth and regeneration. These compositions would be provided in a combined amount effective to accomplish any or all of the foregoing goals. This process may involve providing distinct agent(s) or factor(s) to a cell, tissue or subject at the same time. This may be achieved by treating the cell, tissue, or subject with one or more compositions or pharmacological formulation that include two or three agents, or by treating the cell, tissue, or subject with one, two or three distinct compositions or formulations.

Alternatively, the various agents may precede or follow the second (and/or third) agent or treatment by intervals ranging from minutes to weeks. In embodiments where the second agent (and/or third) and the first agent are administered separately, one would generally ensure that a significant period of time did not expire between the time of each delivery, such that the second (and/or third) agent and the first would still be able to exert an advantageously combined effect on the cell, tissue, or subject. In such instances, it is contemplated that one would treat the cell, tissue, or subject with multiple modalities within about 12-24 hr of each other and, more preferably, within about 6-12 hr of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. It also is conceivable that more than one administration of the first and/or the second and/or the third agent will be desired. Of particular use in combination with SOX2 in the methods disclosed herein, the inventors contemplate provision of nerve growth factors. One example is p′75-2, a mutant form of NT3. Some other exemplary factors are discussed below.

Brain-derived neurotrophic factor (BDNF), or abrineurin, is a protein that in humans is encoded by the BDNF gene. BDNF is a member of the neurotrophin family of growth factors, which are related to the canonical nerve growth factor. Neurotrophic factors are found in the brain and the periphery. BDNF was first isolated from pig brain in 1982 by Yves-Alain Barde and Hans Thoenen.

BDNF acts on certain neurons of the central nervous system and the peripheral nervous system, helping to support survival of existing neurons, and encouraging growth and differentiation of new neurons and synapses. In the brain it is active in the hippocampus, cortex, and basal forebrain—areas vital to learning, memory, and higher thinking. BDNF is also expressed in the retina, kidneys, prostate, motor neurons, and skeletal muscle, and is also found in saliva.

BDNF itself is important for long-term memory. Although the vast majority of neurons in the mammalian brain are formed prenatally, parts of the adult brain retain the ability to grow new neurons from neural stem cells in a process known as neurogenesis. Neurotrophins are proteins that help to stimulate and control neurogenesis, BDNF being one of the most active. Mice born without the ability to make BDNF suffer developmental defects in the brain and sensory nervous system, and usually die soon after birth, suggesting that BDNF plays an important role in normal neural development. Other important neurotrophins structurally related to BDNF include NT-3, NT-4, and NGF.

BDNF is made in the endoplasmic reticulum and secreted from dense-core vesicles. It binds carboxypeptidase E (CPE), and disruption of this binding has been proposed to cause the loss of sorting BDNF into dense-core vesicles. The phenotype for BDNF knockout mice can be severe, including postnatal lethality. Other traits include sensory neuron losses that affect coordination, balance, hearing, taste, and breathing. Knockout mice also exhibit cerebellar abnormalities and an increase in the number of sympathetic neurons.

Certain types of physical exercise have been shown to markedly (threefold) increase BDNF synthesis in the human brain, a phenomenon which is partly responsible for exercise-induced neurogenesis and improvements in cognitive function. Niacin appears to upregulate BDNF and tropomyosin receptor kinase B (TrkB) expression as well.

BDNF binds at least two receptors on the surface of cells that are capable of responding to this growth factor, TrkB (pronounced “Track B”) and the LNGFR (for low-affinity nerve growth factor receptor, also known as p′75). It may also modulate the activity of various neurotransmitter receptors, including the Alpha-7 nicotinic receptor. BDNF has also been shown to interact with the reelin signaling chain. The expression of reelin by Cajal-Retzius cells goes down during development under the influence of BDNF. The latter also decreases reelin expression in neuronal culture.

The TrkB receptor is encoded by the NTRK2 gene and is member of a receptor family of tyrosine kinases that includes TrkA and TrkC. TrkB autophosphorylation is dependent upon its ligand-specific association with BDNF, a widely expressed activity-dependent neurotic factor that regulates plasticity and is unregulated following hypoxic injury. The activation of the BDNF-TrkB pathway is important in the development of short-term memory and the growth of neurons.

The role of the other BDNF receptor, p′75, is less clear. While the TrkB receptor interacts with BDNF in a ligand-specific manner, all neurotrophins can interact with the p75 receptor. When the p75 receptor is activated, it leads to activation of NFkB receptor. Thus, neurotrophic signaling may trigger apoptosis rather than survival pathways in cells expressing the p75 receptor in the absence of Trk receptors. Recent studies have revealed a truncated isoform of the TrkB receptor (t-TrkB) may act as a dominant negative to the p75 neurotrophin receptor, inhibiting the activity of p′75, and preventing BDNF-mediated cell death.

BDNF plays a significant role in neurogenesis. BDNF can promote protective pathways and inhibit damaging pathways in the NSCs and NPCs that contribute to the brain's neurogenic response by enhancing cell survival. This becomes especially evident following suppression of TrkB activity. TrkB inhibition results in a 2-3 fold increase in cortical precursors displaying EGFP-positive condensed apoptotic nuclei and a 2-4 fold increase in cortical precursors that stained immunopositive for cleaved caspase-3. BDNF can also promote NSC and NPC proliferation through Akt activation and PTEN inactivation. There have been many in vivo studies demonstrating BDNF is a strong promoter of neuronal differentiation. Infusion of BDNF into the lateral ventricles doubled the population of newborn neurons in the adult rat olfactory bulb and viral overexpression of BDNF can similarly enhance SVZ neurogenesis. BDNF might also play a role in NSC/NPC migration. By stabilizing p35 (CDK5R1), in utero electroporation studies revealed BDNF was able to promote cortical radial migration by −230% in embryonic rats, an effect which was dependent on the activity of the trkB receptor.

Reference sequences for BDNF are NM_001143805 (mRNA) and NP 001137277 (protein).

Noggin, also known as NOG, is a protein that is involved in the development of many body tissues, including nerve tissue, muscles, and bones. In humans, noggin is encoded by the NOG gene. The amino acid sequence of human noggin is highly homologous to that of rat, mouse, and Xenopus (an aquatic-frog genus). Noggin is an inhibitor of several bone morphogenetic proteins (BMPs): it inhibits at least BMP2, BMP4, BMP5, BMP6, BMP7, BMP13, and BMP14.

Noggin is a signaling molecule that plays an important role in promoting somite patterning in the developing embryo. It is released from the notochord and regulates bone morphogenic protein (BMP4) during development. The absence of BMP4 will cause the patterning of the neural tube and somites from the neural plate in the developing embryo. It also causes formation of the head and other dorsal structures.

Noggin function is required for correct nervous system, somite, and skeletal development. Experiments in mice have shown that noggin also plays a role in learning, cognition, bone development, and neural tube fusion. Heterozygous missense mutations in the noggin gene can cause deformities such as joint fusions and syndromes such as multiple synostosis syndrome (SYNS1) and proximal symphalangism (SIM1). SYNS1 is different from SYM1 by causing hip and vertebral fusions. The embryo may also develop shorter bones, miss any skeletal elements, or lack multiple articulating joints.

Increased plasma levels of Noggin have been observed in obese mice and in patients with a body mass index over 27. Additionally, it has been shown that Noggin depletion in adipose tissue leads to obesity. The secreted polypeptide noggin, encoded by the NOG gene, binds and inactivates members of the transforming growth factor-beta (TGF-beta) superfamily signaling proteins, such as bone morphogenetic protein-4 (BMP4). By diffusing through extracellular matrices more efficiently than members of the TGF-beta superfamily, noggin may have a principal role in creating morphogenic gradients. Noggin appears to have pleiotropic effects, both early in development as well as in later stages.

A study of a mouse knockout model tracked the extent to which the absence of noggin affected embryological development. The focus of the study was the formation of the ear and its role in conductive hearing loss. The inner ear underwent multiple deformations affecting the cochlear duct, semicircular canal, and otic capsule portions. Noggin's involvement in the malformations was also shown to be indirect, through its interaction with the notochord and neural axis. The kinking of the notochord and disorientation of the body axis results in a caudal shift in the embryonic body plan of the hindbrain. Major signaling molecules from the rhombomere structures in the hindbrain could not properly induce inner ear formation. This reflected noggin's regulating of BMP as the major source of deformation, rather than noggin directly affecting inner ear development.

Specific knockout models have been created using the Cre-lox system. A model knocking out Noggin specifically in adipocytes has allowed to elucidate that Noggin also plays a role in adipose tissue: its depletion in adipocytes causes alterations in the structure of both brown and white adipose tissue, along with brown fat dysfunction (impaired thermogenesis and (3-oxidation) that results in dramatic increases of body weight and percent body fat that causes alterations in the lipid profile and in the liver; the effects vary with gender.

Noggin proteins play a role in germ layer-specific derivation of specialized cells. The formation of neural tissues, the notochord, hair follicles, and eye structures arise from the ectoderm germ layer. Noggin activity in the mesoderm gives way to the formation of cartilage, bone and muscle growth, and in the endoderm noggin is involved in the development of the lungs.

Early craniofacial development is heavily influenced by the presence of noggin in accordance with its multiple tissue-specific requirements. Noggin influences the formation and growth of the palate, mandible and skull through its interaction with neural crest cells. Mice with a lack of NOG gene are shown to have an outgrowth of the mandible and a cleft palate. Another craniofacial related deformity due to the absence of noggin is conductive hearing loss caused by uncontrolled outgrowth of the cochlear duct and coiling.

Recently, several heterozygous missense human NOG mutations in unrelated families with proximal symphalangism (SYM1) and multiple synostoses syndrome (SYNS1) have been identified; both SYM1 and SYNS1 have multiple joint fusion as their principal feature, and map to the same region on chromosome 17 (17q22) as NOG. These mutations indicate functional haploinsufficiency where the homozygous forms are embryonically lethal.

Reference sequences for Noggin are NM_001143805 (mRNA) and NP 001137277 (protein).

Neurotrophin-3 (NT3) is a protein that in humans is encoded by the NTF3 gene. NT3 is a neurotrophic factor in the NGF (Nerve Growth Factor) family of neurotrophins. It is a protein growth factor which has activity on certain neurons of the peripheral and central nervous system; it helps to support the survival and differentiation of existing neurons, and encourages the growth and differentiation of new neurons and synapses. NT-3 was the third neurotrophic factor to be characterized, after nerve growth factor (NGF) and BDNF (Brain Derived Neurotrophic Factor).

Although the vast majority of neurons in the mammalian brain are formed prenatally, parts of the adult brain retain the ability to grow new neurons from neural stem cells; a process known as neurogenesis. Neurotrophins are chemicals that help to stimulate and control neurogenesis. NT3 is unique in the number of neurons it can potentially stimulate, given its ability to activate two of the receptor tyrosine kinase neurotrophin receptors (TrkC and TrkB—see below).

NT3 binds three receptors on the surface of cells which are capable of responding to this growth factor:
TrkC is apparently the “physiologic” receptor, in that it binds with greatest affinity to NT3.
NT3 is capable of binding and signaling through a TrkC-related receptors called TrkB.
NT3 also binds a second-receptor type besides Trk receptors, called the LNGFR (for “low affinity nerve growth factor receptor).

Reference sequences for NT3 are NM_001102654 (mRNA) and NP_001096124 (protein).

### 4. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute specifically contemplated modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

Animals. Wild-type C57BL/6J mice (JAX: 000664; RRID: IMSR_JAX:000664) and the following transgenic mice were purchased from the Jackson Laboratory: Rosa-YFP (JAX: 006148; RRID: JAX:006148) (Srinivas et al., 2001), Rosa-tdT (JAX: 007914; RRID: JAX:007914) (Madisen et al., 2010), Sox2^{f/f} (JAX: 013093; RRID: IMSR_JAX:013093) (Shaham et al., 2009), Pdgfra-CreER^{TM} (JAX: 018280; RRID: IMSR_JAX:018280) (Kang et al., 2010), Ascl1-CreER^{T2} (JAX: 012882; RRID: JAX:012882) (Kim et al., 2011), Nes-CreER^{T2} (JAX: 016261; RRID: IMSR_JAX:016261) (Lagace et al., 2007), Foxj1-CreER^{T2} (JAX: 027012; RRID: IMSR_JAX:027012) (Muthusamy et al., 2018; Ren et al., 2017), Rosa-TVAg mouse line (RphiGT, JAX: 024708; RRID: IMSR_JAX:024708) (Takatoh et al., 2013). Both adult male and female mice at 2 months of age and older were used for all experiments unless otherwise stated. All mice were housed under a controlled temperature and a 12-h light/dark cycle with free access to water and food in the animal facility. Sample sizes were empirically determined. Animal procedures and protocols were approved by the Institutional Animal Care and Use Committee at UT Southwestern or Indian University School of Medicine.

Spinal Cord Injuries. Adult mice were anesthetized with a cocktail of ketamine and xylazine. A laminectomy was performed at the indicated spinal segments. A crush injury was carried out by holding a pair of forceps (ZHTG Instrument, China) with a 0.1-mm spacer for 30 seconds, whereas a stab-wound injury was created by injecting a 33-gauge, 18-degree-beveled needle (Hamilton, Reno, Nev.; Cat #22033, customized) into the spinal cord and holding in position for 5 min. A contusion injury was produced by using the IH impactor (Precision Systems and Instrumentation, Lexington, Ky.; Model IH-0400) with a 1-mm tip and a force of 50 kdyne. The C5 dorsal hemisection was created by using a VibraKnife attached to the Louisville Injury System Apparatus (Louisville Impactor System, Louisville, Ky.) (Al-Ali et al., 2017), which can achieve 0.01 mm cutting accuracy (Zhang et al., 2013). The blade was 1.2-mm wide and was advanced 1.2 mm ventrally from the dorsal surface of the cord, extending beyond the central canal. Such a lesion completely transected the entire dorsal corticospinal tract and lateral corticospinal tract on both sides, which are critical for forelimb dexterous and general locomotor function. After surgery, animals were returned to their home cages and received manual bladder expression twice daily until the return of reflexive bladder controls.

Lentivirus Preparation and Intraspinal Injections. A synthetic promoter from the human NG2 gene was subcloned into the lentiviral CS-CDF-CG-PRE vector to create the hNG2-GFP plasmid. Candidate genes were then sub-cloned by PCR into the hNG2-GFP vector at the AgeI and XhoI sites. Lentivirus generation and titer determination were prepared as previously described (Su et al., 2014; Wang et al., 2016). 2-3 μL of lentivirus (0.5-2×10⁹ pfu/mL) was manually injected with a Hamilton syringe and a 33-gauge, 18-degree-beveled needle into the spinal parenchyma at the indicated spinal cord levels. Injections were performed at a rate of 0.3 μL/min. Following injections, the needle was held at the injection site for another 3 min and then slowly withdrawn within one min. For behavioral experiments with C5 dorsal hemisections, a total of 4 stereotaxic viral injections (1-2×10⁹ pfu/ml) were made at 0.5 mm rostral and caudal to the incision to target the NG2 glia at the edge of the lesion. At each distance, bilateral injections (0.5 μl/site) were made according to the following coordinates: mediolateral (ML): 0.5 mm to the midline, and dorsoventral (DV): two injections at 0.4 mm and 0.8 mm each from the dorsal surface of the spinal cord.

Tamoxifen and BrdU Administration. Tamoxifen (T5648, Sigma; Cat #T5648) was dissolved in a mixture of ethanol and sesame oil (1:9 by volume) at a concentration of 40 mg/mL and injected intraperitoneally at a daily dose of 1 mg/10 g body weight for 5-7 days. Proliferating cells were labelled in vivo through administration with BrdU (B5002, Sigma; Cat #B5002; 0.5 g/L) in drinking water for the indicated durations.

Monosynaptic Tracing with Engineered Rabies Virus. The EnvA-pseudotyped RVdG-eGFP rabies virus was obtained from the Vector Core at the Salk Institute. One μl diluted rabies virus (1.6×10⁵ TU/ml) was stereotaxically injected into the previously operated spinal cord location 5-6 months post lentivirus injection. The brain, the brainstem, and the DRG-attached spinal cords were isolated 6 days following rabies virus injection. Series of 60-μm coronal brain/brainstem sections or horizontal spinal cord sections were then collected for confocal analysis.

Immunohistochemistry. Mice were sacrificed with CO₂ overdose and sequentially perfused with ice-cold phosphate-buffered saline (PBS) and 4% (w/v) paraformaldehyde (PFA) in PBS. Whole brains and spinal cords were carefully dissected out, post-fixed overnight with 4% PFA at 4° C., and cryoprotected with 30% sucrose in PBS for 48 h at 4° C. Transverse sections or horizontal sections of a 1.5-cm segment of the spinal cord spanning the injection/injury sites were collected on a cryostat (Leica) set at 20 μm thickness. Coronal brain sections were collected at 60 μm thickness by using a sliding microtome (Leica). The immunostaining procedure was conducted as previously described (Wang et al., 2016). Images were collected on a Zeiss LSM 700 confocal microscope; image-stitching was performed in ZEN software (ZEISS) (RRID: SCR 013672). For cell number counts, every 10th serial 20-μm-thick spinal cord section and a total of 8-10 sections per animal were processed for staining. Confocal images were taken with a 20× objective and the ImageJ program (RRID:SCR 003070) was used for cell counts. The total cell counts from all the processed sections were then multiplied by 10 to arrive the estimated cell number per animal. The ImageJ program was also used for fluorescence intensity analysis.

Behavior Assessments. All behavior experiments were conducted in a separate institution in a randomized and blinded fashion. Animals were randomized into groups and treated with letter-coded viruses. Experimenters were blinded to the identity of the treatments during the behavioral tests. Upon completion of the experiments, mouse groups were analyzed and then decoded. The grid-walking test was employed to assess sensorimotor coordination of the limbs. The test was performed according to a previously established protocol (Al-Ali et al., 2017; Wu et al., 2017). Briefly, 39 animals were pretrained by walking on a wire grid (10 mm×10 mm) for 3 times with each lasting for 3 min each day for 3 days before the experiment. Then they were tested for 3 min at the predetermined experimental time points. The percentage of forepaw drops below the grid plane was calculated from the recorded videos.

Astroglial Scar Analysis. Sagittal sections at 20 μm were cut using a cryostat. Astrocytes were identified by immunostaining for GFAP. 8-12 sagittal spinal cord sections, containing the reactive astroglia, spaced 200 μm were chosen for analysis using the Neurolucida system (MicroBrightfield, Inc.; RRID: SCR_001775). Images were all auto-stitched using the Neurolucida software. Briefly, the area of reactive astroglial scar was manually contoured in each section per every case, and all contours could be 3D reconstructed by cases using the Neurolucida software. Computational analysis of the astroglial scar, including the volume and surface area, were conducted using the Neurolucida software.

Quantification and Statistical Analysis. Data are presented as mean±SEM. Statistical analysis for histological data was performed by homoscedastic two-tailed Student's t-test. Two-way ANOVA and Tukey's post hoc multiple comparisons were used for behavioral data. One-way ANOVA and Tukey's post hoc multiple comparisons were applied for group analysis. A p value <0.05 was considered significant. Significant differences are indicated by *p<0.05, **p<0.01, ***p<0.001, and ****p<0.0001.

### Example 2—Results

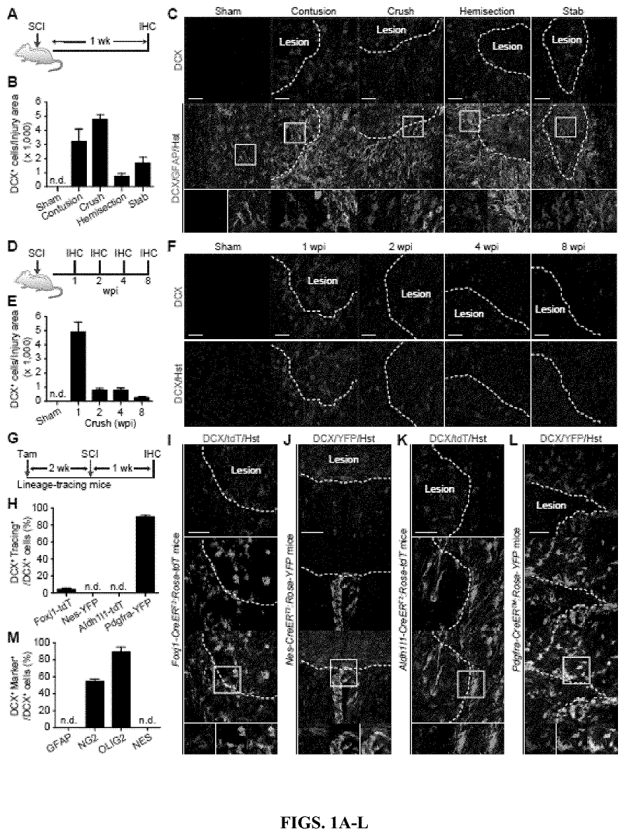

SCI induces neurogenic reprogramming of NG2 glia. The inventors first examined cellular responses to SCI by staining for DCX, a well-established molecular marker for immature neurons during adult neurogenesis (Couillard-Despres et al., 2005; Kempermann et al., 2004; Knoth et al., 2010). Consistent with previous reports (Juhasova et al., 2015; Su et al., 2014), DCX was not detectable in the spinal cord of sham-operated adult mice (FIGS. 1A-C). The thoracic T7-9 level of the spinal cord was then injured through crush. When examined at one-week post-injury (wpi), approximately 5,000 DCX⁺ cells were clearly observed surrounding the GFAP⁺ lesion sites (FIGS. 1A-C). The inventors also detected about 740-3,200 DCX⁺ cells at 1 wpi in other SCI models induced by contusion, hemisection, or stab-wound (FIGS. 1A-C). These cells often intermingled with GFAP⁺ reactive astrocytes surrounding but not far away from the lesion sites (FIG. 1C). A time-course analysis showed that the number of DCX⁺ cells drastically decreased at 2 wpi and subsequent later time points (FIGS. 1D-F).

To determine the cell origin of these injury-induced DCX⁺ cells, the inventors first traced the lineage of ependymal cells lining the central canal, since they were previously reported to show cellular plasticity and exhibit characteristics of neural stem cells following injury (Johansson et al., 1999). These cells were genetically labeled in a tamoxifen-dependent fashion in adult Foxj1-CreER^{T2};Rosa-tdTomato (tdT) mice (FIG. S1A) (Muthusamy et al., 2018; Ren et al., 2017). Two weeks post tamoxifen injection, the inventors injured the T7-9 spinal cord with crush and conducted immunohistochemistry at 1 wpi (FIG. 1G). Although many DCX⁺ cells intermingled with tdT⁺ cells around the lesion site, only about 4.5% of DCX⁺ cells were traced with tdT (FIGS. 1H-I). They also traced nestin-positive (NES⁺) ependymal cells lining the central canal in Nes-CreER^{T2};Rosa-YFP mice (FIGS. S1B-C) (Lagace et al., 2007), since they were reported to show neural stem cell potential after injury (Meletis et al., 2008). However, none of the crush-induced DCX⁺ cells were YFP⁺ surrounding the central canal and lesion site (FIGS. 1H-J). Together, these results indicate that ependymal cells are unlikely the cell origin for injury-induced DCX⁺ cells.

Astrocytes show plasticity following injury and they can be reprogrammed into neuroblasts and neurons in both the adult mouse brain and spinal cord (Gascon et al., 2016; Grande et al., 2013; Guo et al., 2014; Liu et al., 2015; Niu et al., 2015; Niu et al., 2013; Su et al., 2014; Torper et al., 2013; Wang et al., 2016). These cells and their derivatives were traced in adult AldhIl1-CreER^{T2};Rosa-tdT mice after tamoxifen administration (Madisen et al., 2010; Srinivasan et al., 2016). Crush injury was performed 2 weeks later at the T7-9 spinal cord. When analyzed at 1 wpi, many DCX⁺ cells were consistently detected in the injury site, though none of them were traced with tdT (FIGS. 1H-K). The inventors finally traced NG2 glia and their progeny by using the Pdgfra-CreER^{TM};Rosa-YFP mice (Kang et al., 2010; Srinivas et al., 2001). Unexpectedly, approximately 89.8% of crush-induced DCX⁺ cells were labeled with YFP (FIGS. 1H-L). Consistent with this result, immunohistochemistry further revealed that about 89.3% and 54.5% of DCX⁺ cells expressed OLIG2 and NG2, respectively (FIG. 1M, FIGS. S1D-E). In contrast, none of the injury-induced DCX⁺ cells were co-labeled with GFAP or NES (FIGS. 1C and 1M, FIG. S1F). Together, these results indicate that SCI unveils a neurogenic potential of resident NG2 glia.

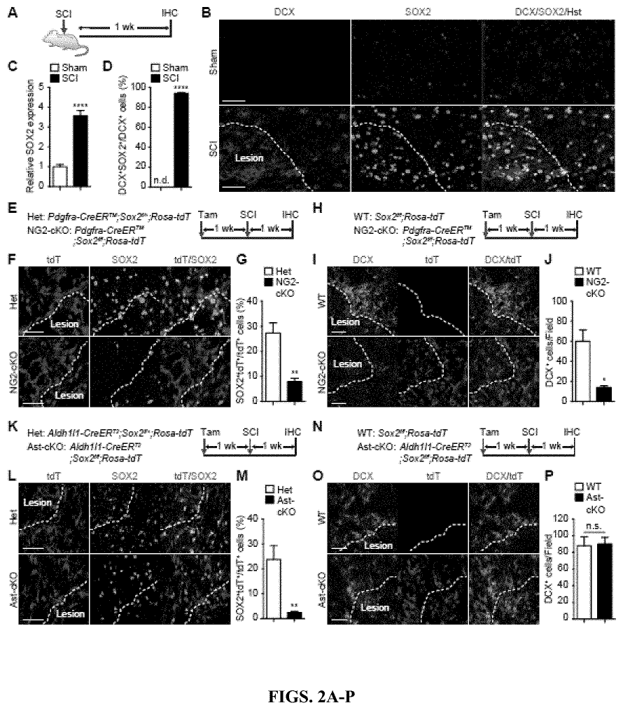

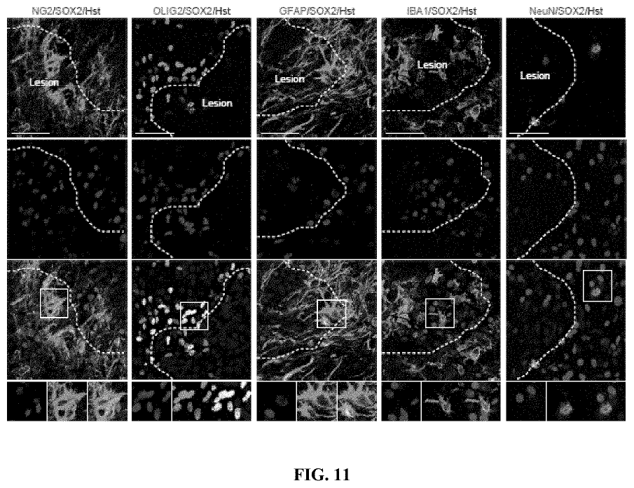

SOX2 is required cell-autonomously for SCI-induced reprogramming of NG2 glia. To understand how SCI induces cell reprogramming, the inventors focused on SOX2, a stem cell factor essential for neurogenesis and neural development (Pevny and Nicolis, 2010). It is induced in glial cells of the adult brain and spinal cord following injury (Chen et al., 2019; Lee et al., 2013) and also required for injury-induced activation of adult cortical astrocytes (Chen et al., 2019). Consistent with these results, the immunohistochemistry showed a 3.6-fold increase of both the number of SOX2⁺ cells and the intensity of SOX2 expression in each cell surrounding the lesion site of the adult mouse spinal cord at 1 wpi (FIGS. 2A-C). Cell type analysis further revealed that SOX2 was mainly detected in astrocytes and NG2 glia but not microglia and neurons (FIG. 11). Most intriguingly, nearly all (94%) of the SCI-induced DCX⁺ cells co-expressed SOX2 (FIG. 2D).

To determine the role of SOX2 in SCI-induced DCX⁺ cells, the inventors took a conditional knockout approach. Sox2 was deleted in NG2 glia after tamoxifen treatment of adult Pdgfra-CreER^{TM};Sox2^{f/f};Rosa-tdT mice. One week later, contusion SCI was introduced and the mice were analyzed on the following week (FIG. 2E).

Immunohistochemistry indicated a 3.4-fold reduction of the number of SOX2-expressing tdT⁺ cells in the lesion sites when compared to their controls (FIGS. 2F-G). Correspondingly, the number of SCI-induced DCX⁺ cells reduced 76.5% in Sox2-deleted mice (FIGS. 2H-J). By using the AldhIl1-CreER^{T2};Sox2^{f/f},Rosa-tdT mice, the inventors similarly examined whether SOX2 in adult astrocytes play any role for SCI-induced DCX⁺cells. Tamoxifen treatment led to an about 10-fold reduction of SOX2-expressing astrocytes in the lesion sites (FIGS. 2K-M). In contrast, the number of SCI-induced DCX⁺ cells remained unchanged after Sox2-deletion in these astrocytes (FIGS. 2N-P). Together, these results show that SOX2 is required cell-autonomously for SCI-induced reprogramming of resident NG2 glia into DCX⁺ cells.

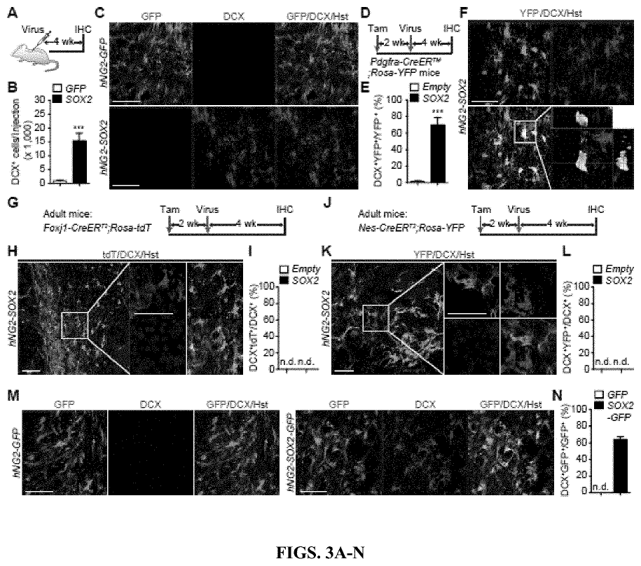

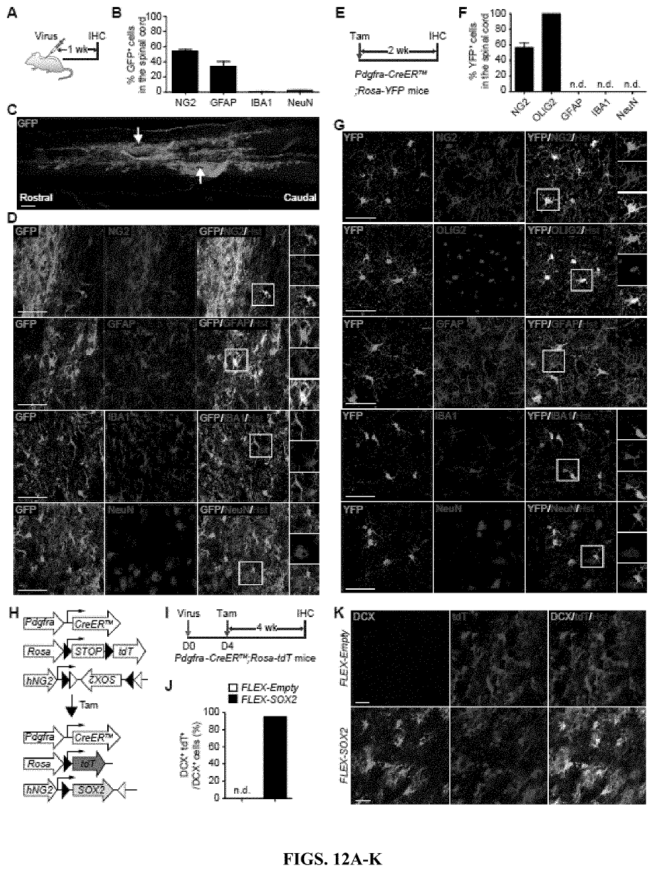

Elevated SOX2 is sufficient to drive neurogenic reprogramming of NG2 glia. These above results suggest that SCI-induced upregulation of SOX2 plays a critical role for generation of DCX⁺ cells from NG2 glia. To determine whether ectopic SOX2 alone was also sufficient to reprogram NG2 glia, the inventors targeted these cells by using a human NG2 promoter (hNG2) in lentivirus. Cell type analysis of the hNG2-GFP reporter in adult mouse spinal cord showed that 54% of GFP⁺ cells expressed NG2, while the remaining cells were GFAP⁺ astrocytes (33.9%) and a few IBA1⁺ microglia (0.7%) and NeuN⁺ neurons (2.3%) (FIGS. 12A-D). They then injected the hNG2-SOX2 virus into the adult spinal cord and analyzed at 4 weeks post virus-injection (wpv) (FIG. 3A), a time point at which very few injury-induced DCX⁺ cells were detectable (FIG. 1E). Excitingly, ectopic SOX2 induced approximately 15,000 DCX⁺ cells surrounding the injection area, whereas less than one thousand DCX⁺ cells were found in the control group (FIGS. 3B-C).

To confirm NG2 glia as the cell origin for SOX2-induced DCX⁺ cells, the inventors employed a genetic lineage tracing approach by using the Pdgfra-CreER^{TM};Rosa-YFP mice. Consistent with previous results (Kang et al., 2010), cell type analysis showed that about 57% and 100% of YFP⁺ cells expressed NG2 and OLIG2, respectively, while markers for GFAP⁺ astrocytes, IBA1⁺ microglia, and NeuN⁺ neurons were non-detectable (FIGS. 12E-G). These mice were injected with hNG2-SOX2 virus 2 weeks post tamoxifen administration and analyzed after another 4 weeks (FIG. 3D). Approximately 70% of SOX2-induced DCX⁺ cells were labeled with YFP (FIGS. 3E-F). To further confirm SOX2 was sufficient to induce DCX⁺ cells from NG2 glia, the inventors used the Cre-dependent hNG2-FLEX-SOX2 virus so that SOX2 expression was restricted to NG2 glia after tamoxifen treatment of the adult Pdgfra-CreER^{TM};Rosa-tdT mice (FIG. 12H). When examined at 4 weeks post tamoxifen administration, 94.8% of SOX2-induced DCX⁺ cells were traced by tdT (FIGS. 12I-K). They also examined the contribution of ependymal cells lining the central canal and found that none of the SOX2-induced DCX⁺ cells were genetically traced in either Foxj1-CreER^{T2};Rosa-tdT (FIGS. 3G-I) or Nes-CreER^{T2};Rosa-YFP mice (FIGS. 3J-L). The reprogramming efficiency was then determined by using the co-expressed GFP in hNG2-SOX2-IRES-GFP virus. About 64% of the GFP⁺ cells expressed DCX⁺, indicating that SOX2 induced efficient reprogramming of NG2 glia (FIGS. 3M-N). Together, these results reveal that ectopic SOX2 alone can robustly reprogram endogenous NG2 glia toward DCX⁺ cells in the adult mouse spinal cord.

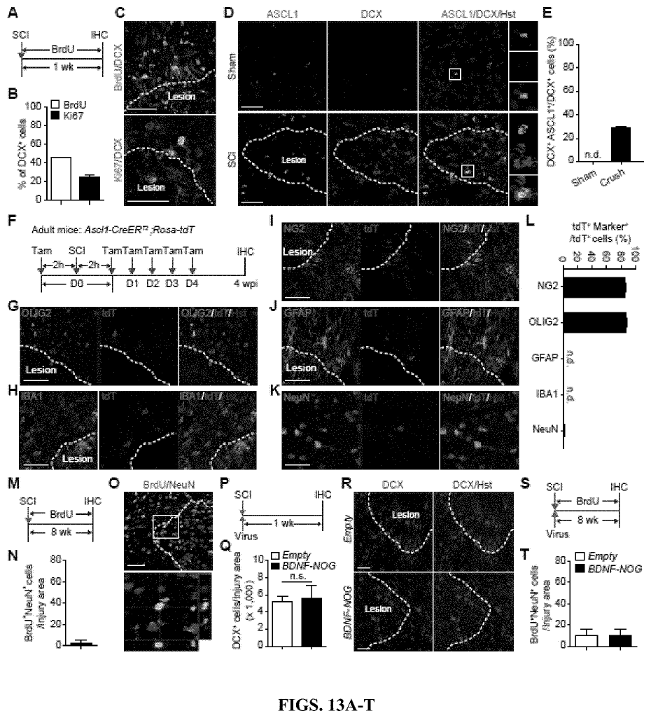

SCI-reprogrammed NG2 glia fail to generate mature neurons. DCX⁺ neuroblasts proliferate during the early stage of adult neurogenesis (Kempermann et al., 2004). The inventors analyzed SCI-induced DCX⁺ cells through BrdU incorporation, which was administered for 1 week in drinking water immediately following crush injury (FIG. 13A). About 45.2% of DCX⁺ cells incorporated BrdU when examined at 1 wpi (FIGS. 13B-C). Immunostaining also showed 25.1% of SCI-induced DCX⁺ cells expressed Ki67, an endogenous marker for cells in active cell cycles (FIGS. 13B-C). ASCL1, a master regulator expressed in neural progenitors and critical for neuronal differentiation and adult neurogenesis (Kim et al., 2011; Raposo et al., 2015), was detected in 28.6% of SCI-induced DCX⁺ cells (FIGS. 13D-E). To investigate the fate of SCI-induced ASCL1⁺ cells, the inventors traced them in adult Ascl1-CreER^{T2};Rosa-tdT knockin mice after 6 tamoxifen-treatments immediately pre- and post-SCI (FIG. 13F). Although only a few cells were traced when examined at 4 wpi, cell type analysis showed that tdT⁺ cells were predominantly co-labeled with NG2 (85.2%) or OLIG2 (86.9%), whereas other cell type-specific markers were rarely observed (FIGS. 13G-L).

To more broadly examine whether SCI induced any newly mature neurons, the inventors treated adult mice immediately following crush injury with BrdU in drinking water for 8 weeks (FIG. 13M). However, only 0-8 BrdU⁺NeuN⁺ cells were detected around the lesions (FIGS. 13N-O). Such a low number of mature neurons might be due to the lack of neurotrophic factors in the adult injured spinal cord. They then injected lentivirus immediately following SCI to express BDNF and NOG, which were previously shown to promote long-term neuronal survival and maturation in the adult mouse brain and spinal cord (Cho et al., 2007; Niu et al., 2013; Wang et al., 2016). Neither virus injections nor the expression of BDNF-NOG influenced the number of SCI-induced DCX⁺ cells (FIGS. 13P-R). When examined at 8 wpi/wpv, however, the inventors still only observed 0-20 BrdU⁺NeuN⁺ neurons in the whole injured spinal cord area (FIGS. 13S-T). Together, these results indicate that SCI only induces transient phenotypic switch of NG2 glia to DCX⁺ cells, which eventually fail to become mature neurons.

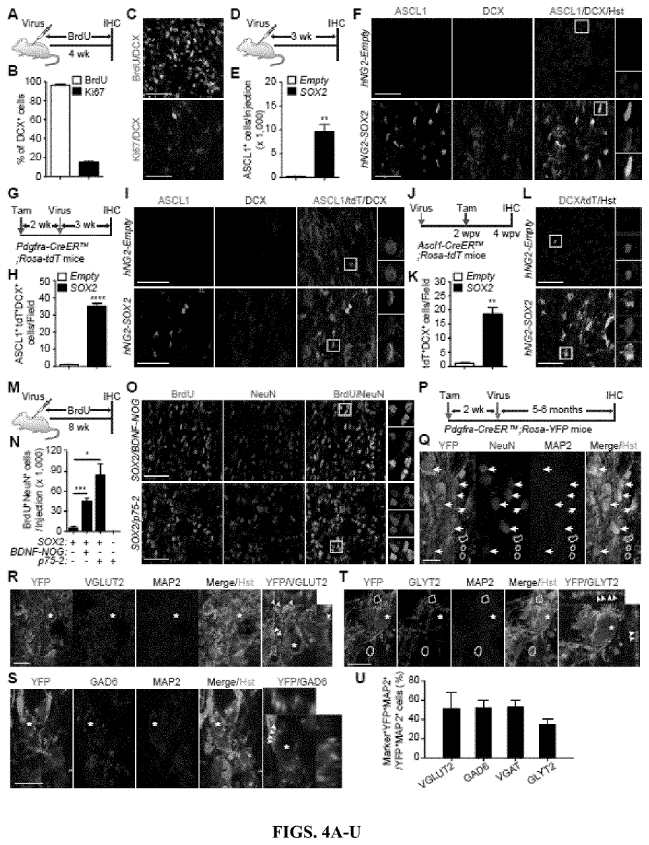

Elevated SOX2 is required for NG2 glia to produce mature neurons. Since ectopic SOX2 was more robust than SCI alone in inducing DCX⁺ cells from NG2 glia (FIG. 3), the inventors next investigated in detail the underlying cellular process. When examined at 4 weeks post injection of hNG2-SOX2 virus, 96% and 15% of SOX2-induced DCX⁺ cells could be respectively labeled by BrdU and Ki67 (FIGS. 4A-C), indicating that they passed through a proliferative progenitor/neuroblast state. Correspondingly, ectopic SOX2 induced nearly 10,000 ASCL1⁺ cells, many of which were also labeled with DCX

(FIGS. 4D-F). By using the adult Pdgfra-CreER^{TM};Rosa-tdT mice, The inventors confirmed that NG2 glia were the cell origin for these induced ASCL1⁺ cells (FIGS. 4G-I). Similarly, they verified that SOX2-induced ASCL1⁺ cells gave rise to DCX⁺ cells by using the Ascl1-CreER^{T2};Rosa-tdT lineage-tracing mice (FIGS. 4J-L). Together, these results clearly indicate that SOX2-induced DCX⁺ cells from NG2 glia pass through an ASCL1⁺ progenitor stage. Such a feature of induced DCX⁺ cells resembles endogenous neuroblasts during adult neurogenesis in the mammalian brain (Kempermann et al., 2004; Kim et al., 2011).

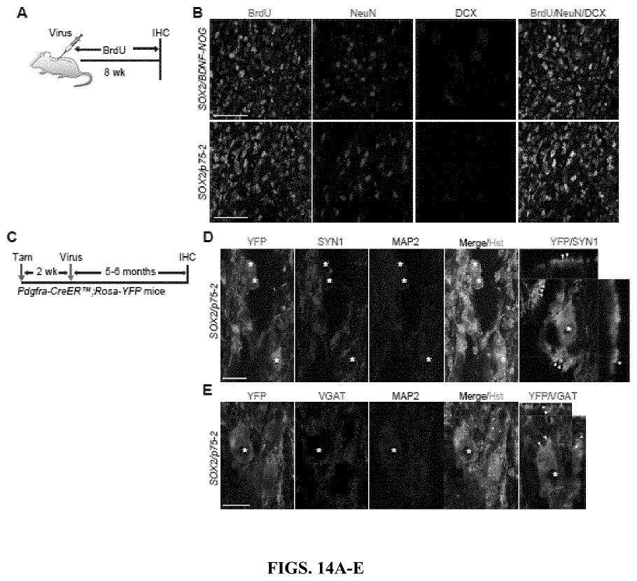

The inventors next investigated whether SOX2-induced DCX⁺ cells could become mature neurons, which were traced by BrdU (FIG. 4M). Excitingly, SOX2 alone induced nearly 5,000 BrdU⁺NeuN⁺ neurons (FIGS. 4N-O). The inclusion of BDNF-NOG increased the number of new neurons to more than 44,000. The inventors also examined p′75-2, a mutant form of neurotrophic factor NT3, which has a reduced affinity to p75NTR and can greatly enhance cell survival and axonal growth after SCI (Enomoto et al., 2013). Interestingly, p75-2 resulted in approximately 84,000 new BrdU⁺NeuN⁺ neurons per injection (FIGS. 4N-O). On the other hand, DCX⁺ cells were rarely observed at this later time point (FIGS. 14A-B), consistent with the notion that DCX⁺ neuroblasts are transient cell states during adult neurogenesis (Kempermann et al., 2004).

The subtypes of SOX2-induced neurons from NG2 glia were then determined through genetic lineage tracing in the spinal cord of adult Pdgfra-CreER^{TM};Rosa-YFP mice (FIG. 4P). These neurons could be clearly identified by the co-expression of YFP and the mature neuronal marker NeuN and MAP2 in both the white and gray matter (FIG. 4Q). The presynaptic marker SYN1 was also robustly detectable, indicating potential synaptic connections with other neurons (FIGS. 14C-D). A survey of markers for neuronal subtypes showed an approximately equal ratio of excitatory (VGLUT2⁺) and inhibitory (GAD6⁺ or VGAT⁺) neurons (FIGS. 4R, 4S, 4U, FIG. 14E). Some of the inhibitory neurons also expressed GLYT2, a marker for glycinergic neurons (FIGS. 4T-U). Together, these data indicate that NG2 glia can be coerced to produce diverse neuronal subtypes in the adult mouse spinal cord.

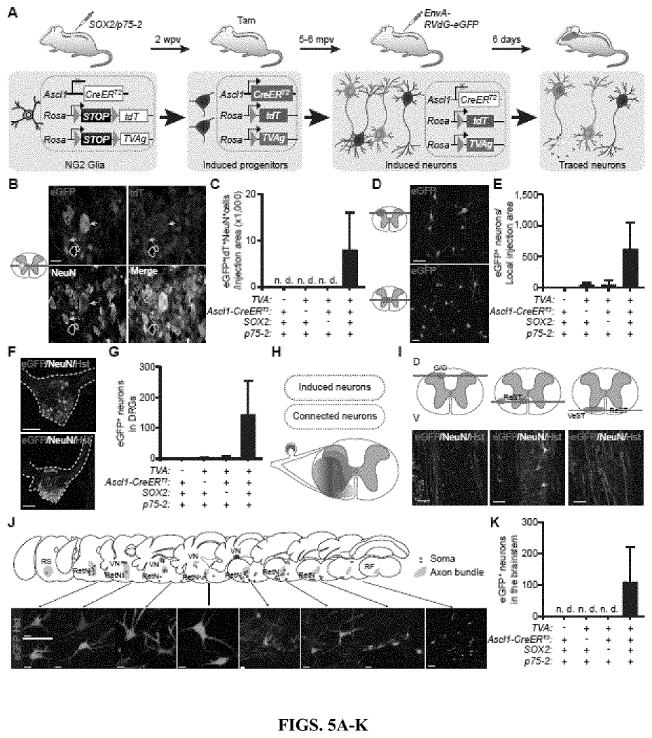

NG2 glia-derived neurons form trans-synaptic connections. Neuron-neuron connections are the cellular basis for function. The inventors examined monosynaptic connections of NG2 glia-derived neurons by using a tracing method based on a recombinant rabies virus (Osakada and Callaway, 2013; Vivar et al., 2012; Wickersham et al., 2007). The EnvA-pseudotyped RVdG-eGFP rabies virus lacks the envelope glycoprotein (G-deleted) but expresses the reporter eGFP. The receptor TVA and rabies glycoprotein (RVg) are expressed in a Cre-dependent manner in the mouse line Rosa-TVAg (also known as RphiGT) (Takatoh et al., 2013). To specifically target the NG2 glia-derived neurons, the inventors used the knockin allele of Ascl1-CreER^{T2} mice (Kim et al., 2011), since ASCL1 is uniquely induced during the neurogenic process (FIGS. 4D-L). They created the Ascl1-CreER^{T2};Rosa-tdT;Rosa-TVAg triple knockin mouse line, such that TVAg is exclusively expressed in the induced tdT⁺ neurons after tamoxifen treatment during the early neurogenic process (FIG. 5A). TVA expression in the induced but not endogenous neurons allows RVdG-eGFP virus to enter the cells. The simultaneously expressed rabies glycoprotein (g) allows retrograde spreading of the viral particles to other neurons with direct presynaptic contacts with the induced neurons (FIG. 5A).

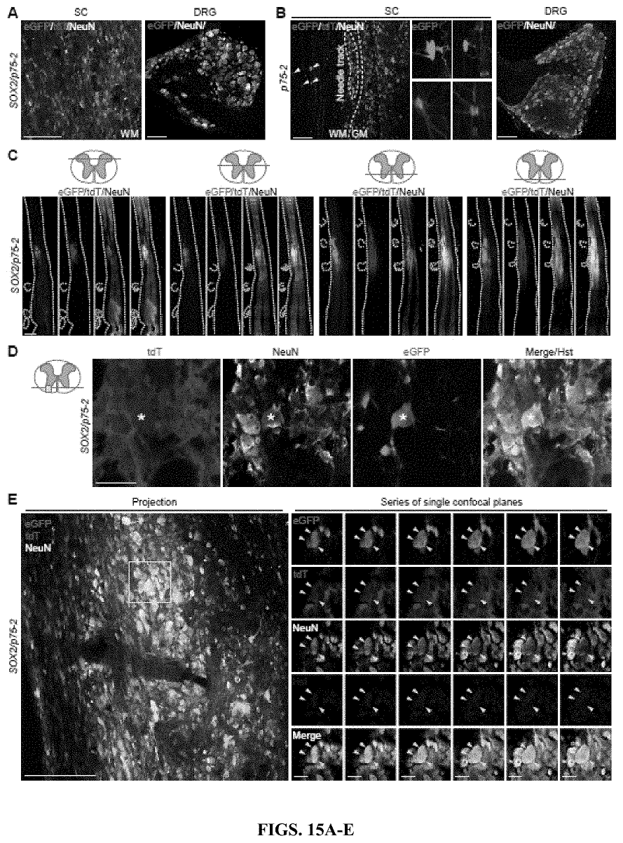

The specificity of the EnvA-pseudotyped RVdG-eGFP rabies virus was confirmed in Ascl1-CreER^{T2};Rosa-tdT mice, which lacked the TVA receptor expression. These mice were injected with lentiviruses expressing SOX2/p75-2, treated with tamoxifen at 2 wpv, injected with the recombinant rabies at 5-6 months post virus injection (mpv), and analyzed after 6 more days. Despite numerous SOX2-induced tdT⁺ cells surrounding the injected spinal cord, none of them were infected with the recombinant rabies, indicated by a lack of eGFP⁺ cells (FIG. 15A). The inventors also examined the recombinant rabies in Ascl1-CreER^{T2};Rosa-tdT;Rosa-TVAg mice that were injected with the control lentivirus p75-2 alone. Only a few tdT⁺ cells with glia morphology were labeled by eGFP around the virus-injected spinal cord areas (FIGS. 15B-C). eGFP⁺ cells were not observed in other regions including the adjacent dorsal root ganglia (DRGs) of these control mice (FIG. 15B; DRG).

The inventors therefore applied this recombinant rabies system to map the presynaptic connections of NG2 glia-derived neurons, which were induced by SOX2/p75-2 in the T7 spinal cords of adult Ascl1-CreER^{T2};Rosa-tdT;Rosa-TVAg mice (FIG. 5A). Horizontal sections of the spinal cords showed robust induction of new tdT⁺NeuN⁺ neurons that were only found in the injected spinal areas including the white and gray matters (FIG. 5B, FIG. 15C). Some of these cells were transduced with the recombinant rabies virus, indicated by coexpression of eGFP, tdT, and NeuN (FIGS. 5B-C, FIGS. 15D-E). Such neurons were not observed in control groups (FIG. 5C). The triple positive neurons could serve as “starter” cells, from which RVdG-eGFP virus could then transmit retrogradely to first order neurons. Many of these first order eGFP⁺ neurons were found throughout the gray matter especially surrounding the injected area in the ipsilateral side (FIGS. 5D-E), although some of them were also in the rostral and caudal side. Interestingly, some neurons in the ipsilateral sides of the DRGs were also traced with eGFP, indicating monosynaptic connections with induced neurons in the spinal cord (FIGS. 5F-H, FIG. 15C). Furthermore, some axon bundles in different horizontal levels of the white matter were labeled with strong eGFP signals (FIG. 5I). They mainly represent the ascending gracile/cuneate tract and the descending reticulospinal and vestibulospinal tracts, respectively. Examinations of serial cross sections through the brain and brainstem showed eGFP⁺ somas were mainly localized in the reticular nuclei and the vestibular nuclei (FIGS. 5J-K). Together, these data indicate that NG2 glia-derived neurons can make monosynaptic connections with propriospinal neurons and those located in the brainstem and DRGs that form the ascending and descending pathways.

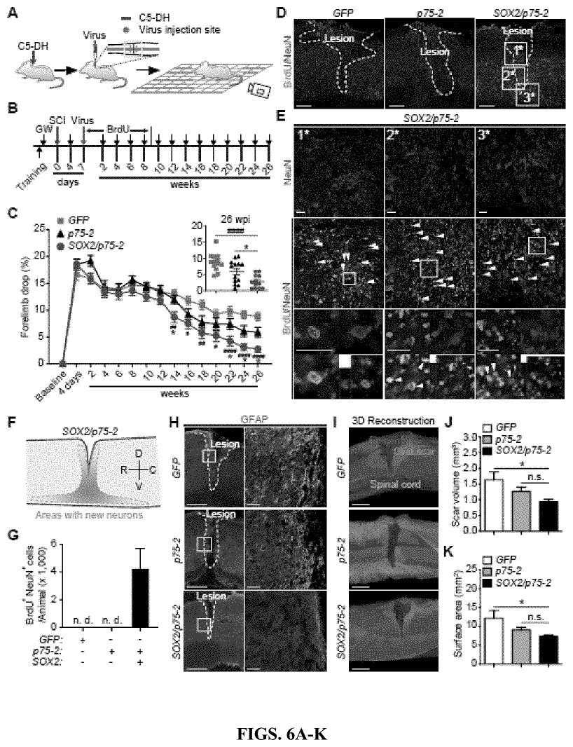

Reprogramming of NG2 glia reduces scar and promotes functional recovery following SCI. The inventors next examined the biological function of reprogramming NG2 glia in a SCI model created by dorsal hemisection at the 5^{th} cervical spina cord level (C5-DH; FIG. 6A) (Zhang et al., 2013). This SCI model was selected since it creates a clearly defined injury penumbra that allows precise virus injections. Furthermore, cervical injuries represent over half of the total SCIs in human patients especially young individuals (Norenberg et al., 2004).

Behavior experiments were conducted in a randomized and blinded fashion. Three groups of viruses (GFP, p75-2, and SOX2/p75-2) were produced and individually coded with a randomized letter. Experimental wildtype mice were pretrained and tested for the grid-walking paradigm before SCI and 4 days following SCI (FIG. 6B). One week post C5-DH, mice were randomized into three groups and injected with the coded viruses (FIGS. 6A-B). The identity of the injected viruses was blinded to the experimenters. Newly born neurons were traced through BrdU in drinking water for 8 weeks. Mice were then behaviorally examined at the indicated time points (FIG. 6B). Upon completion of the experiments, mouse groups were analyzed and then decoded.

The grid-walking paradigm, which requires mice to accurately place their paws on the bars while walking on the grid, examines both basic and skilled locomotion (Al-Ali et al., 2017; Sedy et al., 2008). Successfully walking on the grid depends more on the movement of shoulder and elbow. Both forelimbs were examined in this experiment. Statistical analyses through two-way ANOVA showed significant time- and treatment-dependent effects (FIG. 6C; F(14,454)=176.5 and p<0.0001 for time-dependent effect; F(2,35)=4.299 and p=0.0214 for treatment-dependent effect). Tukey's post-hoc multiple comparisons test revealed that, when compared to either of the control groups, the SOX2/p75-2 group performed significantly better starting at 14 weeks post lesion (SOX2/p75-2 vs. GFP: ^{##}p=0.0015 at 14 wks, ^{#}p=0.0140 at 16 wks, ^{##}p=0.0013 at 18 wks, ^{#}p=0.02223 at 20 wks, ^{####}p<0.0001 at 22 wks, 24 wks and 26 wks; SOX2/p75-2 vs. p75-2: *p=0.0261 at 14 wks, *p=0.0346 at 22 wks, *p=0.0488 at 24 wks, and *p=0.0383 at 26 wks).

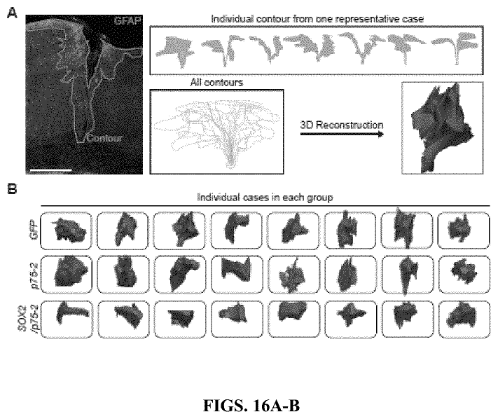

Upon completion of behavioral tests, spinal cords were collected for histological analyses. The inventors first confirmed generation of new neurons in this SCI model. These new neurons, indicated by BrdU⁺NeuN⁺, were mainly distributed surrounding the lesion/injection area of the SOX2/p75-2 group (FIGS. 6D-F). Approximately 4,000 BrdU⁺NeuN⁺ neurons were detected in the group treated with SOX2/p75-2 virus but not in the other two control groups (FIG. 6G). They also examined glial scar by staining GFAP. Astrocytic boundaries were well defined surrounding the lesions (FIG. 6H). Interestingly, 3D reconstructions (FIGS. 16A-B) and quantifications showed that both scar volume and surface area were significantly reduced in SOX2/p75-2 group when compare to the GFP control (FIG. 6I-K; *p=0.024′7 and *p=0.0361, respectively). The p75-2 group also showed moderate but not significant reduction of astrocytic scars (FIGS. 6J-K). These results indicate that reprogramming of NG2 glia leads to generation of new neurons and reduction of glial scars, both of which may contribute to functional improvements post SCI.

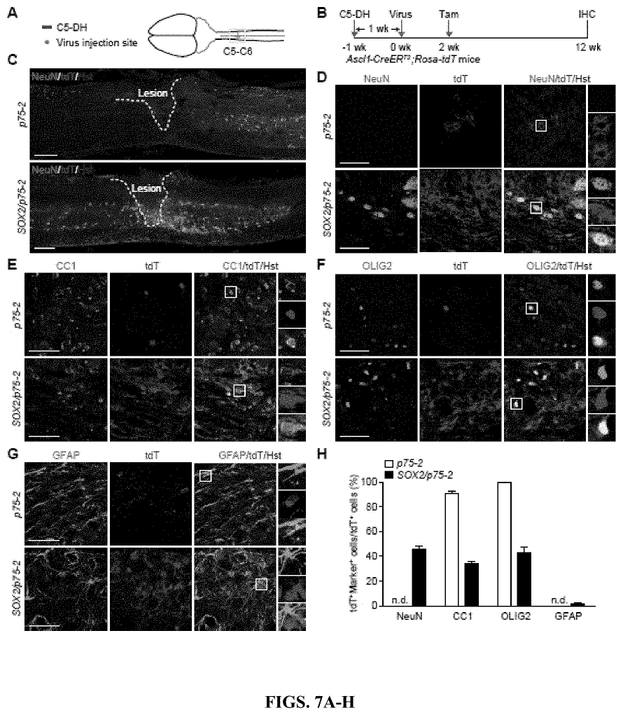

SOX2-reprogrammed NG2 glia are bipotent and generate both neurons and oligodendrocytes. NG2 glia is the cell source for continued oligodendrogenesis in the adult central nervous system (Nishiyama et al., 2016; Tripathi et al., 2010). Reprogramming their fate for neurogenesis may lead to depletion of NG2 glia and disruption of oligodendrogenesis. The inventors examined such a scenario by using the lineage tracing mouse line Ascl1-CreER^{T2};Rosa-tdT, since ASCL1 was uniquely induced by SOX2 from reprogrammed NG2 glia (FIGS. 4D-L). Adult Ascl1-CreER^{T2};Rosa-tdT mice underwent C5-DH injury and were injected with viruses into the surrounding lesion area at 1 wpi (FIGS. 7A-B). These mice were then administered with tamoxifen at 2 wpv and examined at 12 wpv (FIG. 7B). Numerous tdT⁺ cells were detected surrounding the lesion area of mice injected with the SOX2/p75-2 virus (FIG. 7C). In sharp contrast, only a few tdT⁺ cells were sparsely observed in the p75-2 control (FIG. 7C). Cell types were determined by using specific markers and showed that 45.8% of tdT⁺ cells were NeuN⁺ neurons in the SOX2/p75-2 group, whereas such NeuN⁺tdT⁺ cells were not detected in the p75-2 control (FIGS. 7C-D, 7H). Interestingly, 34.6% and 43.5% of SOX2-induced tdT⁺ cells expressed the oligodendrocyte marker CC1 and OLIG2, respectively, while the number of GFAP⁺tdT⁺ cells was minimal (FIGS. 7E-H). On the other hand, the few sparsely distributed tdT⁺ cells in the control p75-2 group all expressed CC1 or OLIG2 (FIGS. 7D-H). Together, these results indicate that SOX2-reprogrammed NG2 glia are bipotent, generating both neurons and oligodendrocytes. As such, oligodendrogenesis is not abolished but maybe even enhanced by SOX2-mediated reprogramming of NG2 glia.

### Example 3—Discussion

Multiple cell types can change their morphology and behavior in response to injury. This study shows that SCI elicits neurogenic potential of NG2 glia. Elevated SOX2 is both required and sufficient for neurogenic reprogramming of NG2 glia, which can eventually lead to functional neurogenesis in the normally non-neurogenic adult mouse spinal cord. The results indicate that the expression level of SOX2 is critically important and a threshold must be overcome to enable NG2 glia to become robustly neurogenic.

NG2 glia, also known as oligodendrocyte precursor cells, are the major proliferative glial cells in the adult central nervous system (Buffo et al., 2008; Kang et al., 2010). They serve as a pool of progenitors to differentiate into oligodendrocytes and a few protoplasmic astrocytes (Kang et al., 2010; Nishiyama et al., 2016; Tripathi et al., 2010). In response to injury, NG2 glia increase their numbers and become a major component of the glial scar (Lytle and Wrathall, 2007; Tripathi and McTigue, 2007). The inventors' finding that SCI-induces robust expression of DCX in these cells also indicate that they may undergo phenotypic reprogramming towards neurogenesis. However, such injury-induced phenotypic reprogramming is transient and incomplete for producing mature neurons. This is consistent with previous reports showing that NG2 glia are not neurogenic (Kang et al., 2010; Tripathi et al., 2010) but rather remain largely undifferentiated and contribute to neuroinflammation and axon regeneration failure after injury (Filous et al., 2014; Hackett and Lee, 2016; Levine, 2016). Reprogramming these cells by SOX2 not only provides new neurons but may also ameliorate the pathological microenvironment, indicated by glial scar reduction. Interestingly, SOX2-induced ASCL1⁺ progenitors from NG2 glia are bipotent, giving rise to both new neurons and oligodendrocytes.

The inventors observed that SCI-induced DCX⁺ cells are not derived from ependymal cells that form the central canal. They traced central canal ependymal cells in both the Foxj1-CreER^{T2};Rosa-tdT and Nes-CreER^{T2};Rosa-YFP mice and failed to detect meaningful DCX expression in the lineage-mapped cells. These results are in agreement with multiple recent reports showing that ependymal cells lack properties of neural stem cells and rarely contribute new cells to the injury site (Muthusamy et al., 2018; Ren et al., 2017; Shah et al., 2018).

Similar to NG2 glia, adult astrocytes become reactive, proliferate, and contribute to glial scar formation following injury (Gotz et al., 2015; Sofroniew and Vinters, 2010). When isolated and cultured in vitro, reactive astrocytes can form neurospheres and gain multilineage differentiation potential (Gotz et al., 2015). Moreover, these cells can be in vivo reprogramed into neurons by multiple transcription factors including SOX2 (Gascon et al., 2016; Grande et al., 2013; Guo et al., 2014; Liu et al., 2015; Niu et al., 2015; Niu et al., 2013; Su et al., 2014; Torper et al., 2013). Notwithstanding, the inventors fail to detect DCX expression in these cells or their derivatives following SCI. This is not because of lack of SOX2 expression, as the results from this study and others show that SOX2 is endogenously expressed in reactive astrocytes (Chen et al., 2019; Su et al., 2014). It could be that the SCI-induced endogenous SOX2 level fails to pass a threshold to enable neurogenic reprogramming of reactive astrocytes, suggesting that different cell types may require different levels of SOX2. Alternatively, other factors in astrocytes maintain their fate in a more stable state. Furthermore, glial cells may also show regional differences in response to injury, as DCX expression is not detected in the injured adult mouse brain cortex that is enriched with reactive glial cells (Heinrich et al., 2014).

It is remarkable that endogenous SOX2 is absolutely required for SCI-induced neurogenic reprogramming of NG2 glia. SOX2 is normally expressed in NG2 glia and can be further up-regulated by SCI (Lee et al., 2013; Zhang et al., 2018). It remains to be determined how the injury signal is transmitted into the nucleus to control SOX2 expression. Although SOX2 can work as a pioneer transcription factor, the requirement of ectopic SOX2 for complete neurogenic reprogramming of NG2 glia suggests that some SOX2-regulated genomic sites may have low binding affinity and that transcription of such genomic sites is required for fate transition. Alternatively, transcriptional outputs of some SOX2 targets may well be determined by the expression levels of SOX2 and sustained high outputs of these targets are essential for fate transition. It should be interesting in the future to examine these scenarios through ATAC-seq, ChIP-seq, and RNA-seq under various conditions. Such investigations will provide significant insights into the molecular mechanisms underlying fate maintenance and reprogramming, which may be manipulated for neural regeneration.

Synaptic connections between neurons are the cellular basis for function. Studies show that propriospinal relay connections around injury sites can mediate pronounced spontaneous functional recovery without regeneration of direct projections from the brain past the lesion (Courtine et al., 2008). An agonist of the neuron-specific K⁺—Cl⁻ co-transporter (KCC2) can restore consistent stepping ability in paralyzed SCI mice through reactivation of dormant relay pathways (Chen et al., 2018). These findings indicate that it is possible to restore function after SCI through relay connections around injury area. The SOX2-induced propriospinal neurons from NG2 glia may well form such relays surrounding the lesions. The transition through an ASCL1⁺ progenitor stage of SOX2-mediated neurogenesis enables us to precisely and specifically map relay connections of new neurons. This is accomplished by using a Cre-dependent recombinant rabies virus in mice harboring knockin alleles of Ascl1-CreER^{T2},Rosa-tdT, and Rosa-TVAg. These results provide evidence that new spinal neurons can be directly targeted by endogenous neurons originating in the brainstem, spinal cord, and DRGs. The presynaptic neurons located in the brainstem mainly form the descending reticulospinal tracts (ReSTs) and vestibulospinal tracts (VeSTs). The ReSTs play a critical role in preparation of movements and postural control, whereas the VeSTs are responsible for initiation of limb and trunk extensor activity (Watson and Harvey, 2009). The DRGs contain cell bodies of sensory neurons that bring information from the periphery to interneurons in the spinal cord and some of those sensory neurons can also bifurcate to form the ascending gracile/cuneate pathways to relay sensory information to the brain. These results indicate that new spinal neurons make presynaptic connections with neurons controlling sensorimotor function. Due to the lack of a robust, specific, and Cre-dependent monosynaptic anterograde tracing method, the immediate postsynaptic connections of the new spinal neurons await to be determined in the future.

Together, these results reveal a cellular and molecular mechanism underlying neural injury-induced cell plasticity, which can be further exploited for adult neurogenesis and relay formation in a region that has largely lost the ability to regenerate. Through expandable progenitors giving rise to both neurons and oligodendrocytes, reprogramming of NG2 glia not only ameliorates the pathological microenvironment but may also provide the much-needed new cells for network reconstruction and functional improvement following injury.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### 6. REFERENCES

The following references, to the extent that they provide exemplary procedural or other details supplementary to those set forth herein, are specifically incorporated herein by reference.
PCT/US14/16905
U.S. Patent Publication No. 20070010831
“Antibodies: A Laboratory Manual,” Cold Spring Harbor Press, Cold Spring Harbor, N.Y., 1988.
Angel et al., Cell, 49:729, 1987a.
Angel et al., Cell, 49:729, 1987b.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Benvenisty and Neshif, Proc. Natl. Acad. Sci. USA, 83:9551-9555, 1986.
Berkhout et al., Cell, 59:273-282, 1989.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chatterjee et al., Proc. Natl. Acad. Sci. USA, 86:9114, 1989.
Chen and Okayama, Mol. Cell Biol., 7(8):2745-2752, 1987.
Choi et al., Cell, 53:519, 1988.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81-90, 1988.
Coupar et al., Gene, 68:1-10, 1988.
Cripe et al., EMBO J., 6:3745, 1987.
Culotta and Hamer, Mol. Cell. Biol., 9:1376-1380, 1989.
Dandolo et al., J. Virology, 47:55-64, 1983.
De Villiers et al., Nature, 312(5991):242-246, 1984.
Deschamps et al., Science, 230:1174-1177, 1985.
Dubensky et al., Proc. Natl. Acad Sci. USA, 81:7529-7533, 1984.
Edbrooke et al., Mol. Cell. Biol., 9:1908-1916, 1989.
Edlund et al., Science, 230:912-916, 1985.
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Foecking and Hofstetter, Gene, 45(1):101-105, 1986.
Fraley et al., Proc. Natl. Acad. Sci. USA, 76:3348-3352, 1979.
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Graham and van der Eb, Virology, 52:456-467, 1973.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101(3):1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hersdorffer et al., DNA Cell Biol., 9:713-723, 1990.
Hirochika et al., J Virol., 61:2599, 1987.
Holbrook et al., Virology, 157:211, 1987.
Horlick and Benfield, Mol. Cell. Biol., 9:2396, 1989.
Horwich et al. J Virol., 64:642-650, 1990.
Huang et al., Cell, 27:245, 1981.
Hug et al., Mol. Cell. Biol., 8:3065-3079, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9(8):3393-3399, 1989.
Kadesch and Berg, Mol. Cell. Biol., 6:2593, 1986.
Kaneda et al., Science, 243:375-378, 1989.
Karin et al., Mol. Cell. Biol., 7:606, 1987.
Katinka et al., Cell, 20:393, 1980.
Kato et al., J Blot Chem., 266(6):3361-3364, 1991.
Kiledjian et al., Mol. Cell. Biol., 8:145, 1988.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kuhl et al., Cell, 50:1057, 1987.
Kunz et al., Nucl. Acids Res., 17:1121, 1989.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Laughlin et al., J. Virol., 60(2):515-524, 1986.
Lebkowski et al., Mol. Cell. Biol., 8(10):3988-3996, 1988.
Lee et al., Nature, 294:228, 1981.
Levinson et al., Nature, 295:79, 1982.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
Markowitz et al., J. Virol., 62:1120-1124, 1988.
McLaughlin et al., J Virol., 62(6):1963-1973, 1988.
McNeall et al., Gene, 76:81, 1989.
Miksicek et al., Cell, 46:203, 1986.
Mordacq and Linzer, Genes and Dev., 3:760, 1989.
Moreau et al., Nucl. Acids Res., 9:6047, 1981.
Muesing et al., Cell, 48:691, 1987.
Muzyczka, Curr. Topics Microbiol. Immunol., 158:97-129, 1992.
Naldini et al., Science, 272(5259):263-267, 1996.
Ng et al., Nuc. Acids Res., 17:601, 1989.
Nicolas and Rubinstein, Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Ornitz et al., Mol. Cell. Biol., 7:3466, 1987.
Palmiter et al., Cell, 29:701, 1982.
Pech et al., Mol. Cell. Biol., 9:396, 1989.
Pelletier and Sonenberg, Nature, 334:320-325, 1988.
Perales et al., Proc. Natl. Acad. Sci. USA, 91(9):4086-4090, 1994.
Perez-Stable and Constantin, Mol. Cell. Biol., 10:1116, 1990.
Physicians Desk Reference.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mot Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Reisman and Rotter, Mol. Cell. Biol., 9:3571, 1989.
Remington's Pharmaceutical Sciences, 15th ed., 1035-1038 and 1570-1580, Mack Publishing Company, PA, 1980.
Resendez Jr. et al., Mol. Cell. Biol., 8:4579, 1988.
Ridgeway, In: Vectors: A Survey of Molecular Cloning Vectors and Their Uses, Rodriguez et al. (Eds.), Stoneham: Butterworth, 467-492, 1988.
Ripe et al., Mot Cell. Biol., 9:2224, 1989.
Rippe et al., Mot Cell Biol., 10:689-695, 1990.
Rittling et al., Nuc. Acids Res., 17:1619, 1989.
Rosen et al., Cell, 41:813, 1988.
Sakai et al., Genes and Dev., 2:1144, 1988.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. 1, 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
The Merck Index, 11th Edition.
Thiesen et al., J. Virology, 62:614, 1988.
Tratschin et al., Mot Cell. Biol., 4:2072-2081, 1984.
Tronche et al., Mot Biol. Med., 7:173, 1990.
Tronche et al., Mol. Cell. Biol., 9:4759, 1989.
Trudel and Constantini, Genes and Dev., 6:954, 1987.
Tur-Kaspa et al., Mol. Cell Biol., 6:716-718, 1986.
U.S. Pat. No. 5,139,941
U.S. Pat. No. 5,580,859
U.S. Pat. No. 5,670,488
U.S. Pat. No. 5,672,681
U.S. Pat. No. 5,994,136
U.S. Pat. No. 6,013,516
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Wagner et al., Proc. Natl. Acad. Sci. USA 87(9):3410-3414, 1990.
Wang and Calame, Cell, 47:241, 1986.
Weber et al., Cell, 36:983, 1984.
Weinberger et al. Mol. Cell. Biol., 8:988, 1984.
Wong et al., Gene, 10:87-94, 1980.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Yang et al., Proc. Natl. Acad. Sci. USA, 87:9568-9572, 1990.
Yutzey et al. Mol. Cell. Biol., 9:1397, 1989.
Zelenin et al., FEBS Lett., 280:94-96, 1991.
Zufferey et al., Nat. Biotechnol., 15(9):871-875, 1997.
Al-Ali et al., (2017). J Neurosci 37, 7079-7095.
Barnabe-Heider et al. (2010). Cell Stem Cell 7, 470-482.
Buffo et al., (2008). PNAS USE 105, 3581-3586.
Chen et al., (2018). Cell 174, 1599.
Chen et al., (2019). Cereb Cortex 29, 54-69.
Cho et al., (2007). J Clin Invest 117, 2889-2902.
Couillard-Despres et al., (2005). European J Neurosci 21, 1-14.
Courtine et al., (2008). Nature Medicine 14, 69-74.
Enomoto et al., (2013). Experimental Neurology 248, 170-182.
Filous et al., (2014). J Neurosci 34, 16369-16384.
Gascon et al., (2016). Cell Stem Cell 18, 396-409.
Gotz et al., (2015). Glia 63, 1452-1468.
Grande et al., (2013). Nature Comm 4, 2373.
Guo et al., (2014). Cell Stem Cell 14, 188-202.
Hackett, A. R., and Lee, J. K. (2016). Frontiers in Neurology 7, 199.
Heinrich et al., (2014). Stem Cell Reports 3, 1000-1014.
Horky et al., (2006). J Comp Neurol 498, 525-538.
Horner et al., (2000). 1 Neurosci. 20, 2218-2228.
Johansson et al., (1999). Cell 96, 25-34.
Juhasova et al., (2015). Cellular and Molecular Neurobiology 35, 57-70.
Kang et al., (2010). Neuron 68, 668-681.
Kempermann et al., (2004). Trends Neurosci 27, 447-452.
Kim et al., (2011). PloS One 6, e18472.
Knoth et al., (2010). PloS One 5, e8809.
Lagace et al., (2007). 1 Neurosci 27, 12623-12629.
Lee et al., (2013). J Neuroscience Res 91, 196-210.
Levine, J. (2016). Brain Research 1638, 199-208.
Lie et al., (2004). Annu Rev Pharmacol Toxicol 44, 399-421.
Liu et al., (2015). J Neurosci 35, 9336-9355.
Lytle, J. M., and Wrathall, J. R. (2007). European J Neurosci 25, 1711-1724.
Madisen et al., (2010). Nature Neuroscience 13, 133-140.
Meletis et al., (2008). PLoS Biol 6, e182.
Muthusamy et al., (2018). Scientific Reports 8, 1766.
Nishiyama et al., (2016). Brain Research 1638, 116-128.
Niu et al., (2015). Stem Cell Reports 4, 780-794.
Niu et al., (2013). Nat Cell Biol 15, 1164-1175.
Norenberg et al., (2004). J Neurotrauma 21, 429-440.
Obernier et al., (2018). Cell Stem Cell 22, 221-234 e228.
Osakada, F., and Callaway, E. M. (2013). Nat Protoc 8, 1583-1601.
Pevny, L. H., and Nicolis, S. K. (2010). Int J Biochem Cell Biol 42, 421-424.
Raposo et al., (2015). Cell Reports 10, 1544-1556.
Ren et al., (2017). Scientific Reports 7, 41122.
Sedy et al., (2008). Neurosci Biobehavioral Rev 32, 550-580.
Shah et al., (2018). Cell 173, 1045-1057 e1049.
Shaham et al., O (2009). Development 136, 2567-2578.
Sofroniew, M. V., and Vinters, H. V. (2010). Acta Neuropathol 119, 7-35.
Srinivas et al., (2001). BMC Developmental Biology 1, 4.
Srinivasan et al. (2016). Neuron 92, 1181-1195.
Su et al., (2014). Nature Communications 5, 3338.
Takatoh et al., (2013). Neuron 77, 346-360.
Torper et al., (2013). PNAS USA 110, 7038-7043.
Tripathi, R., and McTigue, D. M. (2007). Glia 55, 698-711.
Tripathi et al., (2010). J Neurosci 30, 16383-16390.
Vivar et al., (2012). Nature Communications 3, 1107.
Wang et al., (2016). Cell Reports 17, 891-903.
Watson, C., and Harvey, A. R. (2009). Projections from the Brain to the Spinal Cord. In The Spinal Cord (Academic Press), pp. 168-179.
Wickersham et al., (2007). Neuron 53, 639-647.
Wu et al., (2017). PLoS One 12, e0178803.
Yamamoto et al., (2001). Experimental Neurology 172, 115-127.
Zhang et al., (2018). Mol Neurobiol 55, 9001-9015.
Zhang et al., (2013). J Vis Exp, e50030.
Ziv et al., (2006). PNAS USA 103, 13174-13179.

1. A method of reprogramming a non-nerve cell into a neuronal cell comprising contacting said non-nerve cell with a SOX2 agonist.

2. A method of inducing regeneration, repair and/or growth of nerve tissue comprising contacting said a non-nerve cell in nerve tissue with a SOX2 agonist.

3. The method of claim 1, wherein said non-nerve cell is a glial cell or fibroblast.

4. The method of claim 1, wherein said neuronal cell is a neuron.

5. The method of claim 4, wherein said neuron is a brain neuron or a spinal neuron.

6. The method of claim 4, wherein said neuron is an inhibitory neuron or a excitatory neuron.

7. The method of claim 1, wherein SOX2 agonist is a SOX2 protein or an expression construct comprising a SOX2 coding region under the control of a promoter active in mammalian cells.

8. The method of claim 7, wherein said promoter is a tissue specific promoter, such as glial cell or stromal cell specific promoter, including but not limited to NG2, GFAP, or PDGFRA.

9. The method of claim 7, wherein said promoter is a constitutive promoter or an inducible promoter.

10. The method of claim 7, wherein said expression construct is a viral or non-viral expression vector.

11. The method of claim 1, further comprising contacting said non-nerve cell with a neuronal growth factor.

12. The method of claim 11, wherein said neuronal growth factor is brain-derived neurotrophic factor (BDNF), noggin (NOG), or NT3.

13. The method of claim 1, wherein said non-nerve cell is located in a living mammalian subject, such as a human.

14. The method of claim 13, wherein said subject has suffered a nerve injury.

15. The method of claim 14, wherein said nerve injury is a spinal cord injury, traumatic brain injury, a stroke, or a neurodegenerative disease.

16. The method of claim 15, wherein said SOX2 agonist is contacted with said non-nerve cell more than once.

17. The method of claim 15, wherein said neuronal growth factor is contacted with said non-nerve cell more than once.

18. The method of claim 13, further comprising treating said subject with physical therapy or other nerve deficit therapy prior to, at the time of, or post-contacting.


SEQUENCE LISTING (4 sequences)

SEQ ID NO: 1 - DNA, 954 bp, Homo sapiens
  source 1..954: mol_type = other DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 2 - DNA, 960 bp, Mus musculus
  source 1..960: mol_type = other DNA
atgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtga

SEQ ID NO: 3 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 4 - protein, 319 aa, Mus musculus
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM